SEQUENCE LISTING (22 sequences)

SEQ ID NO: 1 - DNA, 20 bp, Homo sapiens
aggccagagcaaacagaaaa

SEQ ID NO: 2 - DNA, 20 bp, Homo sapiens
atggagggtgacgtcttgag

SEQ ID NO: 3 - DNA, 19 bp, Homo sapiens
gaccacacagcccattctg

SEQ ID NO: 4 - DNA, 20 bp, Homo sapiens
tagaggtgcaaaggagcgat

SEQ ID NO: 5 - DNA, 20 bp, Homo sapiens
actgttgcagcatgagttcc

SEQ ID NO: 6 - DNA, 19 bp, Homo sapiens
ctccttggttttggggatg

SEQ ID NO: 7 - DNA, 20 bp, Homo sapiens
agcagctgggactgagtcat

SEQ ID NO: 8 - DNA, 20 bp, Homo sapiens
aagcttgtctggtggctgtt

SEQ ID NO: 9 - DNA, 20 bp, Homo sapiens
gtgcctggagaagttgaagg

SEQ ID NO: 10 - DNA, 20 bp, Homo sapiens
ctgaagctggtcgatttggt

SEQ ID NO: 11 - DNA, 20 bp, Homo sapiens
gagagggttgttcccactga

SEQ ID NO: 12 - DNA, 20 bp, Homo sapiens
aaaggcctgccttacactga

SEQ ID NO: 13 - DNA, 20 bp, Homo sapiens
gatcctgctgctttgctttc

SEQ ID NO: 14 - DNA, 20 bp, Homo sapiens
cacacacacacacaccctga

SEQ ID NO: 15 - DNA, 20 bp, Homo sapiens
ttcaccatgattggagagca

SEQ ID NO: 16 - DNA, 20 bp, Homo sapiens
ctgaactgttggtgggacct

SEQ ID NO: 17 - DNA, 20 bp, Homo sapiens
ctgtcccgaaagaaaaccaa

SEQ ID NO: 18 - DNA, 20 bp, Homo sapiens
ggcataagattccgaagcag

SEQ ID NO: 19 - DNA, 20 bp, Homo sapiens
ggctggtcttactggagcag

SEQ ID NO: 20 - DNA, 20 bp, Homo sapiens
catcagccagaatcccatct

SEQ ID NO: 21 - DNA, 21 bp, Homo sapiens
ctctgctcctcctgttcgaca

SEQ ID NO: 22 - DNA, 20 bp, Homo sapiens
acgaccaaatccgttgactc

(19) United States
(12) Patent Application Publication
(10) Pub. No.: US 2017/0304386 A1
(43) Pub. Date: Oct. 26, 2017

(54) METHOD FOR INHIBITING SURVIVAL, TUMORIGENESIS AND METASTASIS OF CANCER CELLS AND/OR CANCER STEM CELLS

(71) Applicant: Shih-Chieh HUNG, Taipei City (TW)

(72) Inventors: Shih-Chieh HUNG, Taipei City (TW); Han-Shui Hsu, Taipei City (TW)

(73) Assignee: Shih-Chieh HUNG, Taipei City (TW)

(21) Appl. No.: 15/517,885

(22) PCT Filed: Oct. 8, 2015
(86) PCT No.: PCT/CN2015/091452

§ 371 Date: Apr. 7, 2017

Related U.S. Application Data
(60) Provisional application No. 62/061,278, filed on Oct. 8, 2014.

(51) Int. Cl. A61K 38/02; A61K 39/395; C12Q 1/68; A61K 31/00

(52) U.S. Cl. CPC A61K 38/02; C12Q 1/6813; C12Q 1/6806; A61K 39/395; A61K 31/00

(57) ABSTRACT

A new method for preventing tumorigenicity or treating a cancer in a subject includes administering to the subject a Collagen XVII (Col XVII) inhibitor in an amount effective to inhibit and prevent survival, tumorigenesis and metastasis of cancer cells and/or cancer stem cells (CSCs).

### FIELD OF THE INVENTION

The present invention generally relates to a new method for inhibiting and preventing survival, tumorigenesis and metastasis of cancer cells and/or tumor initiation or cancer stem cells (TICs or CSCs) in a subject. In particular, the present invention provides a use of Collagen XVII (Col XVII) inhibitor to TICs in inhibiting and preventing survival, tumorigenesis and metastasis of said tumor initiation or cancer stem cells.

### BACKGROUND OF THE INVENTION

The discovery of tumor initiation or cancer stem cells (TICs) as cancer initiating components has provided an attractive approach to treating cancers. TICs make up a minor population of cancer cells, which possess stem cell properties, such as self-renewal and multi-potency for differentiation. Deregulation of TIC self-renewal is a possible requirement for the development of cancer, and survival of TICs may be responsible for the resistance to cancer therapies and recurrence of tumors. Since TICs are responsible for tumor initiation and treatment resistance, uncovering new characteristics of TICs and inhibiting the signal pathways have rapidly become a novel therapeutic strategy for treating cancer.

Highly pure populations of TICs have been identified in the subpopulations of cells positive for certain distinct surface markers, such as CD133, EpCAM, CD44, CD166 (O'Brien CA, Pollett A, Gallinger S, Dick J E (2007) A human colon cancer cell capable of initiating tumour growth in immunodeficient mice. Nature 445: 106-110; Ricci-Vitiani L, Lombardi D G, Pilozzi E, Biffoni M, Todaro M, Peschle C, De Maria R (2007) Identification and expansion of human colon-cancer-initiating cells. Nature 445: 111-115), or obtained by spheroid condition, a suspension culture in a serum-free medium (Dalerba P, Dylla S J, Park I K, Liu R, Wang X, Cho R W, Hoey T, Gurney A, Huang E H, Simeone D M et al (2007) Phenotypic characterization of human colorectal cancer stem cells. Proc Natl Acad Sci USA 104: 10158-10163). Cancer cells proliferate/differentiate in anchorage-independent conditions, giving rise to clonal spheroids, which can in part recapitulate the primary tumor expression profile (Lee J, Kotliarova S, Kotliarov Y, Li A, Su Q, Donin N M, Pastorino S, Purow B W, Christopher N, Zhang W et al (2006) Tumor stem cells derived from glioblastomas cultured in bFGF and EGF more closely mirror the phenotype and genotype of primary tumors than do serum-cultured cell lines. Cancer Cell 9: 391-403). Contrary to the dogma that epithelial cell survival is anchorage-dependent, previous reports have demonstrated that a small population of normal neural or mammary stem cells when cultured in suspension condition can generate floating spherical colonies (Dontu G, Abdallah W M, Foley J M, Jackson K W, Clarke M F, Kawamura M J, Wicha M S (2003) In vitro propagation and transcriptional profiling of human mammary stem/progenitor cells. Genes Dev 17: 1253-1270; Reynolds B A, Weiss S (1996) Clonal and population analyses demonstrate that an EGF-responsive mammalian embryonic CNS precursor is a stem cell. Dev Biol 175: 1-13; Weiss S, Reynolds B A, Vescovi A L, Morshead C, Craig CG, van der Kooy D (1996) Is there a neural stem cell in the mammalian forebrain? Trends Neurosci 19: 387-393). Although these data together strongly implicate that TICs or normal stem cells may have better suspension survival ability than other cells, there are few, if any, studies investigating specifically whether these cells increased in suspension survival ability and elucidating the underlying mechanisms.

### SUMMARY OF THE INVENTION

This invention is based on the unexpected finding that Collagen XVII (Col XVII) serves as downstream target of S727-phosphorylated STAT3 (^{S727}STAT3) and mediates suspension survival ability via the formation of hemidesmosome structure in cancer stem cells (CSCs).

Accordingly, in one aspect, the present invention provides a method for inhibiting and preventing survival, tumorigenesis and metastasis of cancer cells and/or cancer stem cells (CSCs) in a subject comprising administering the subject with a pharmaceutical composition comprising Collagen XVII (Col XVII) inhibitor to CSCs in an amount effective in inhibiting and preventing the formation of hemidesmosome structures, and the survival, tumorigenesis and metastasis of said cancer stem cells.

In another aspect, the invention provides a use of Col XVII) inhibitor to CSCs for the manufacture of a medicament for inhibiting and preventing the formation of hemidesmosome structures, and the survival, tumorigenesis and cancer cells and/or CSCs.

In the present invention, the Col XVII inhibitor is a component capable of blocking PP2A-^{S727}STAT3-Col XVII pathway, a component capable of inhibiting Col XVII itself, or a component capable of inhibiting ^{S727}STAT3-Col XVII-laminin 5-FAK pathway whereby mediating the formation of hemidesmosome structures, suspension survival and tumor initiation in CSCs. In one embodiment of the present invention, the Col XVII inhibitor is selected from the group consisting of a microRNA (miRNA), a small interfering RNA (siRNA), a chemical agent, an antibody, an inhibitor of enzyme existing in PP2A-^{S727}STAT3-Col XVII pathway, and an inhibitor of enzyme existing in ^{S727}STAT3-Col XVII-laminin 5-FAR pathway.

In further aspect, the present invention also provides a pharmaceutical composition for inhibiting and preventing the formation of hemidesmosome structures, and the survival, tumorigenesis and metastasis of cancer cells and/or cancer stem cells, comprising an effective amount of Col XVII inhibitor.

In still further aspect, the present invention also provides a use of a CSC surface protein or extracellular protein for the manufacture of a kit for preventing tumorigenicity and treating cancer, wherein the CSC surface protein or extracellular protein is Col XVII.

These and other aspects will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

The foregoing summary, as well as the following detailed description of the invention, will be better understood when read in conjunction with the appended drawing. In the drawings:

FIGS. 1a-1f show the characterization of spheroid culture-enriched TICs, followed by FIG. 1a, which representative pictures of sphere formed from CCS and HT29 in spheroid culture under non-adherent or adherent condition, Bar=50 μm, and FIG. 1b, which CCS cancer cells in spheroid culture were subcultured for every 15 days. Quantification of number of CCS spheres for three consequent generations with size over 250 μm for five pictures. FIGS. 1c (Western blot) and 1d (flow cytometry) were analysis for bulk cancer cells (Bulk) and spheroid culture-enriched TICs (SPH). FIG. 1e (left), which immunostaining for Cdx2 of bulk cancer cells, spheroid culture-enriched TICs, and TICs after differentiation in matrigel for one month, Bar=50 μm, FIG. 1e (right), quantification of positive cells was performed with five pictures per sample. FIG. 1f shows the tumourigenicity of bulk cancer cells and spheroid-enriched TICs derived from CCS and HT29 cells. The results are expressed as mean±SD of three independent experiments. Asterisks indicate significant differences determined by one-way ANOVA (b, e), student's t-test (d), and Fisher's exact test (f).

FIGS. 2a-2i show better suspension survival ability of spheroid culture-enriched TICs. FIG. 2a (left), representative pictures of TUNEL staining for CCS, and the FIG. 2a (right), quantification of TUNEL positive cells. FIG. 2b shows the Bulk cancer cells (Bulk) and spheroid culture-enriched TICs (SPH) were cultured in spheroid condition for 24 h, followed by TUNEL assay. FIG. 2c shows ALDH⁻ and ALDH⁺ cells which isolated from HCW primary liver metastasis colorectal cancer cells were cultured in spheroid condition for 24 h, followed by TUNEL assay. The flow cytometry histogram shows the expression of ALDH⁺ cells in the HCW primary liver metastasis cancer cells and fresh colorectal cancer specimen (Dotted line: a control with 5 μM of DEAB; solid line: ALDH expression), and the methodology of flow cytometry cell sorting in HCW cells, which P6 is the ALDH⁻ sorting area and P5 is the ALDH⁺ sorting area. FIG. 2d shows the Bulk cancer cells (Bulk) and spheroid culture-enriched TICs (SPH) were cultured in spheroid condition for 10 h, followed by western blot analysis, respectively. FIG. 2e shows the bulk cancer cells (Bulk) and spheroid culture-enriched TICs (SPH) were cultured in dishes, coated with Poly-HEMA (6 mg/ml) containing growth medium and 0.5% methylcellulose (MC) in the absence or presence of 5% matrigel (MG) for 24 h. FIG. 2e (left), representative pictures of TUNEL staining for CCS, Bar=50 μm, and FIG. 2e (right), quantification of TUNEL positive cell. FIG. 2f (top), representative pictures of anchorage-independent growth assay in soft agar for CCS, and FIG. 2f (bottom), quantification of colonies per well. FIG. 2g shows the CD133⁻ and CD133⁺ cells isolated from culture-enriched TICs of CCS were cultured in spheroid condition for 24 hours, followed by TUNEL assay. The results are expressed as mean±SD of three independent experiments. Asterisks indicate significant differences determined by one-way ANOVA. FIG. 2h shows the CCS cancer cells were labeled with PKH, followed by culture in spheroid condition for 15 days. Spheroids were subjected to TUNEL staining and immunostaining for β-tubulin and cleaved-caspase 3 (C3). FIG. 2i shows the CCS bulk cancer cells (Bulk) and spheroid culture-enriched TICs (SPH) were subjected to in vivo suspension condition in the absence or presence of 5% matrigel (MG) for 24 hours. FIG. 2i (left), representative pictures of TUNEL staining, and the FIG. 2i (right), quantification of TUNEL positive cells. Quantification of TUNEL assay was performed with five pictures for each sample. The results are expressed as mean±SD of three independent experiments. Asterisks indicate significant differences determined by student's t-test or one-way ANOVA. Bar=20 μm.

FIGS. 3a-3i shows the activation of STAT3 at S727 which mediates the suspension survival ability of spheroid-enriched TICs. FIGS. 3a-3c show the bulk cancer cells (Bulk) and spheroid culture-enriched TICs (SPH) were cultured in spheroid condition for 10 hours, followed by western blot assay of total protein (see FIG. 3a) or fractionated protein (N, nucleus and C, cytoplasm) (see FIG. 3b) and immunostaining followed by examination with a confocal microscope (CCS) (see FIG. 3c). FIG. 3d shows the CCS cancer cells were labeled with PKH, followed by culture in spheroid condition for 15 days. Spheroids were subjected to immunostaining followed by examination with a confocal microscope. FIG. 3e show the spheroid cultures of CCS overexpressed with control shRNAs (CTR) or different STAT3 shRNAs, si(1) and si(2), were suspended and cultured in spheroid condition for 24 hours, followed by TUNEL assay. FIG. 3e (top), western blot analysis of STAT3 knockdown efficiency, and FIG. 3e (bottom), quantification of TUNEL positive cells. FIGS. 3f and 3h show the spheroid culture cells overexpressed with control plasmids (CTR) or S727A point-mutated STAT3 (SA). FIGS. 3g and 3i show the bulk cancer cells overexpressed with control plasmids (CTR) or S727E point-mutated STAT3 (SE) without or with Y705F mutation (YFSE). FIGS. 3f and 3g show the cells were cultured in spheroid condition for 24 hours, followed by TUNEL assay. FIGS. 3f and 3g (top), western blot analysis, and FIGS. 3f and 3g (bottom), quantification of TUNEL positive cells. FIGS. 3h and 3i show the cells derived from CCS were subjected to in vivo suspension condition for 24 hours, followed by TUNEL assay. The results are expressed as mean±SD of three independent experiments. Asterisks indicate significant differences determined by student's t-test and one-way ANOVA. Bar=20 μm.

FIGS. 4a-4m shows the Col XVII up-regulated by S727-phosphorylated STAT3 mediates the suspension survival ability of enriched TICs. FIG. 4a (top), western blot for spheroid culture of day 0, 5 and 15, and FIG. 4a (bottom), the schematic representation of CCS cell preparation for investigating gene expression profiles and subsequent data analysis. FIG. 4b shows the up-regulated genes in CCS-derived TICs were distributed in cancer and cell death categories through Gene Ontology (GO) and pathway analysis. FIG. 4c shows the CCS cancer cells before (day 0), and 5 or 15 days after spheroid culture were harvested for mRNA isolation, followed by quantitative RT-PCR analysis. FIGS. 4d-4e show the quantitative RT-PCR for HT29 bulk cancer cells (Bulk) and spheroid culture-enriched TICs (SPH), and the Bulk cancer cells (Bulk) and spheroid culture-enriched TICs (SPH) were cultured in spheroid condition for 10 h, followed by FIG. 4d (western blot), and FIG. 4e (immunostaining) followed by examination with a confocal microscope (CCS). FIG. 4f shows the cells overexpressed with control plasmids (CTR) or S727A point-mutated STAT3 (SA) were assayed for mRNA and protein levels. FIG. 4g shows the cells overexpressed with control plasmids (CTR) or S727E point-mutated STAT3 (SE) were assayed for mRNA and protein levels. FIGS. 4h-4i show the spheroid cultures or bulk cancer cells expressing S727E point-mutated STAT3 were overexpressed with control shRNAs (CTR) or different Col17a1 shRNAs, si(1) and si(2). Cells were cultured in spheroid condition for 24 hours, followed by TUNEL assay, including. FIGS. 4h-4i (top) showing western blot of Col17a1 knockdown efficiency, and FIGS. 4h-4i (bottom) showing quantification of TUNEL positive cells. FIG. 4j shows the genomic organization of the region flanking the promoter region of Col17a1 (top panel) and the schematic representation of the pEZX-PG04-Col17a1 reporter construct. Transcription start site (TSS). FIG. 4k (left), ChIP analysis of bulk cancer cells expressing S727E point-mutated STAT3. The chromatin was incubated either without antibodies, with an anti-FLAG antibody or with an isotype IgG antibody. Fragments of the 8^{th} binding site in the Col17a1 promoter (C8) were amplified by PCR. FIG. 4k (right), quantification of DNA binding with quantitative RT-PCR. Input, 4% of total input lysate. FIG. 4l shows the spheroid culture-enriched TICs (SPH) expressing S727A point-mutated STAT3 were employed to analyze the role of S727 phosphorylation in activating Col17a1 promoter. FIG. 4m shows the mutational analysis of the 8^{th} binding site in the Col17a1 promoter. Reporter constructs containing wild-type (WT) and box 8 mutations were employed to analyze the importance of this site in mediating activation by STAT3. The results are mean±SD of three independent experiments. Asterisks indicate significant differences determined by student's t-test and one-way ANOVA. Bar=20 μm.

FIGS. 5a-5k show the Col XVII-mediated suspension survival ability depends on laminin 5 and FAK activation. FIGS. 5a and 5b show the Bulk cancer cells (Bulk) and spheroid culture-enriched TICs (SPH) were cultured in spheroid condition for 10 h, followed by FIG. 5a (western blot assay), and FIG. 5b (immunofluorescence) followed by examination with a confocal microscope. FIG. 5c shows the spheroid culture of cells overexpressed with control plasmids (CTR) or S727A point-mutated STAT3 (SA) were assayed for protein levels. FIG. 5d shows the Bulk cancer cells overexpressed with control plasmids (CTR) or S727E point-mutated STAT3 (SE) were assayed for protein levels. FIGS. 5e-5f show the spheroid cultures of cells overexpressed with control shRNAs (CTR) or different Col17a1 shRNAs, si(1) and si(2) were assayed for mRNA and protein levels (FIG. 5e), and immunostaining (CCS)(FIG. 5f) followed by examination with a confocal microscope. FIG. 5g (top), western blot analysis for spheroid culture-enriched TICs expressing control shRNAs (CTR) or Col17a1 shRNAs, si(1) or si(2), treated with 50 μg/ml cycloheximide in the presence or absence of 10 μM MG132 for 6, 12, 24, and 36 hours. FIG. 5g (bottom), graphical representation of the relative band intensities of laminin 5 protein. The percentage of initial intensities of laminin 5 protein in cell extracts normalized with respect to β-tubulin amounts was plotted against time. FIG. 5h shows the Bulk cancer cells (Bulk) and spheroid culture-enriched TICs (SPH) were cultured in spheroid condition for 10 hours, followed by western blot assay. FIG. 5i shows the western blot analysis for spheroid culture-enriched TICs expressing control shRNAs (CTR) or Col17a1 shRNAs, si(1) or si(2). FIG. 5j shows the TICs (SPH) overexpressed with control plasmids (CTR), S727A point-mutated STAT3 (SA) were assayed for protein levels. FIG. 5k shows the Bulk cells overexpressed with control plasmids (CTR), S727E point-mutated STAT3 (SE) were assayed for protein levels. Bar=20 μm.

FIGS. 6a-6e show the spheroid culture-enriched TICs possess hemidesmosome-like plaques and survive under suspension conditions. FIG. 6a shows the aggregate of HT29 bulk cancer cells formed by hanging drop culture (Bulk) and sphere formed in HT29 spheroid culture (SPH) were subjected to immunofluorescence followed by examination with a confocal microscope. FIG. 6b shows the TEM image of wild-type hemidesmosomes in spheroid culture-formed sphere (arrow), Bar=0.2 μm. FIG. 6c shows the cells in FIG. 6a were cultured in spheroid condition for 24 hours, followed by Live/Dead analysis. FIG. 6d shows the spheres formed in spheroid culture of HT29 cells transfected with scrambled (Scr) or shRNA against Col17a1 (siCol17a1) or laminin 5 (siLaminin 5) were subjected to immunofluorescence followed by examination with a confocal microscope, and spheroid culture for 24 hours followed by Live/Dead analysis. FIG. 6e shows the spheres formed by spheroid culture treated with trypsin or chymotrypsin treatment for 30 min were subjected to spheroid culture for 24 hours followed by Live/Dead analysis, Bar=20 μm.

FIGS. 7a-7m show the PP2A regulates STAT3 activation at S727 and mediates suspension survival ability in TICs. FIG. 7a shows the Bulk cancer cells (Bulk) and spheroid culture-enriched TICs (SPH) were cultured in spheroid condition for 10 hours, followed by western blot assay. FIG. 7b shows the spheroid culture of PKH-labeled CCS cells was assayed for immunostaining. FIG. 7c shows the western blot analysis for CCS bulk cancer cells treated with 0, 0.1, 0.5, and 1 nM OA for 1 hour or 0, 5, 10, and 15 nM CA for 30 min. FIG. 7d shows the western blot analysis for CCS spheroid culture-enriched TICs treated with 50 nM ceramide C6 for 24 hours. FIG. 7e shows the western blot analysis for immunoprecipitated PP2A or STAT3 complex of CCS bulk cancer cells. FIG. 7f shows the spheroid culture-enriched TICs expressing control plasmid (CTR) or wild-type (WT) PP2A were subjected to spheroid condition for 24 hours, FIG. 7f (top), western blot analysis; FIG. 7f (bottom), quantification of TUNEL positive cells. FIG. 7g shows the Bulk cancer cells expressing control plasmid (CTR) or dominant negative (DN) PP2A were subjected to spheroid condition for 24 hours, FIG. 7g (top), western blot analysis; FIG. 7g (bottom), quantification of TUNEL positive cells. FIG. 7h shows the cells in FIG. 7f were subjected to in vivo suspension condition for 24 hours, followed by TUNEL assay. FIG. 7i shows the cells in FIG. 7g were subjected to in vivo suspension condition for 24 hours, followed by TUNEL assay. FIG. 7j shows the enriched TICs derived from non-adherent culture and ALDH⁺ cancer cells increase in PP2A/^{S727}STAT3/Col XVII pathway. FIG. 7j (left) shows the Bulk cancer cells (Bulk) and spheroid culture-enriched TICs (SPH) derived from non-adherent culture were cultured in spheroid condition for 10 hours, followed by western blot analysis, and FIG. 7j (right) shows the western blot analysis for ALDH⁻ and ALDH⁺ cells isolated from HCW primary liver metastasis cancer cells and fresh colorectal cancer specimen. FIG. 7k shows the Bulk cancer cells (Bulk) and spheroid culture-enriched TICs (SPH) were cultured in spheroid condition for 24 hours, followed by western blot analysis. The FIG. 7l shows the quantification of TUNEL positive cells. FIG. 7m shows the Bulk cancer cells (Bulk) and spheroid culture-enriched TICs (SPH) were cultured in spheroid condition for 10 hours, followed by western blot analysis. The results are expressed as mean±SD of three independent experiments. Asterisks indicate significant differences determined by student's t-test. Bar=20 μm.

FIGS. 8a-8f show the suspension survival ability determines tumourigenicity and corresponds to clinical tumour staging and survival. FIG. 8a shows the tumourigenicity of bulk cancer cells expressing DN PP2A and S727E point-mutated STAT3 with or without snRNA against Col17a1 or TICs over-expressing WT PP2A, S727A point-mutated STAT3, and snRNA against Col17a1. FIGS. 8b and 8c show the culture-enriched TICs (SPH) of HT29 and ALDH⁺ cells isolated from HCW primary liver metastasis cancer cells and fresh cancer cells were cultured in spheroid condition for 24 hours with treatment of STAT3 inhibitor (S3I-201, 50 μM) or 1:25 dilution of the 0.25 mg/ml anti-Col XVII antibody, followed by TUNEL assay. (Ab-1, Abcam; Ab-2, Pierce). FIG. 8d shows the tumourigenicity of enriched TICs (HT29) directly treated with STAT3 inhibitor (S3I-201, 50 μM), 1:25 dilution of the 0.25 mg/ml anti-Col XVII antibody or STAT3 inhibitor intravenously (S3I-201, 5 mg/kg) post injection of TICs. FIG. 8e shows the expression of pS727STAT3 and Col XVII in primary colorectal tumours was detected by immunohistochemical staining of tissue array slides. FIG. 8e (top), representative pictures of positive staining of colorectal tumour tissues (different stages) are shown, FIG. 8e (bottom), quantification and the correlation between protein expression and tumour stages was subsequently determined. The results are expressed as percentage of DAB positive cells and every single plot represents a patient. The median is shown as a single black line. FIG. 8f shows the survival curves of 150 colorectal cancer patients with or without up-regulated pS727STAT3 and Col XVII expression (calculated using the Kaplan-Meier method). The expression of pS727STAT≧40% or Col XVII 50% was regarded as positive. Asterisks indicate significant differences determined by Fisher's exact test, one-way ANOVA, and log-rank test.

FIGS. 9a-9e shows the expression of Col XVII and laminin 5 by TICs in human lung cancer-related malignant pleural effusion and the requirement of the Col XVII/laminin 5 pathway in malignant lung pleural effusion induced by enriched TICs from A549 cells. FIG. 9a shows the cells derived from human malignant pleural effusion natively formed spheres, which could be maintained in spheroid culture for weeks, while cells derived from benign pleural effusion of congestive heart failure did not form spheres even cultured under spheroid conditions. FIGS. 9b and 9c show the immunofluorescence revealed that tumour cells in human malignant pleural effusion adopted spheroid morphology and expressed CD44, a surface marker for lung cancer stem cells, Col XVII and laminin 5. FIG. 9d shows the immunofluorescence revealed the expression of Col XVII and laminin 5 by enriched TIC derived from A549 cells. FIG. 9e shows the ability of enriched TICs from A549 cells to form malignant pleural effusion is dependent on the Col XVII-laminin 5 pathway. Micro-CT was performed before sacrifice to demonstrate intrapleural and extrapulmonary tumour growth. FIG. 9e (left), gross images of the pleural cavities of mice received injections of indicated numbers of bulk or enriched TICs from A549 cells, and FIG. 9e (right), the ratio of injections with indicated numbers of cells from different conditions to form malignant pleural effusion. Bar=20 μm.

FIGS. 10A-10F show that A549 and CL1-1 cells in spheroid culture demonstrated the features of cancer stem-like cells (CSC) and upregulation of epithelial-to-mesenchymal transition (EMT) markers, as well as increased invasion and migration properties in vitro. FIG. 10A shows A549 and CL1-1 cells showed increased sphere formation when cultured in non-adherent culture dishes and spheroid medium for 12 days compared to those cultured in non-spheroid medium. A549 cells cultured in adherent dishes and DMEM with 10% FBS were used as controls and no sphere formation was noted. FIG. 10B shows the result of Western blot analysis of Oct-4, Nanog, and Sox2 in A549 and CL1-1 cells. FIG. 10C shows the result of Western blot analysis of EMT markers, including Snail, E-cadherin, N-cadherin, fibronetin and vimentin. FIG. 10D shows the result of the migration assay. FIG. 10E shows the result of the invasion assay. FIG. 10E shows the result of the wound healing assay at 0 and 24 hours. The results are expressed as mean±standard deviation of three independent experiments. Asterisks indicate significant differences as determined by student's t-test or one-way ANOVA. Bars indicate 50 μm.

FIGS. 11A-11G show the results of the microarray analysis, which shows upregulation of Col XVII and laminin-5 in lung cancer stem-like cells (CSC) and knockdown of Col XVII reduced sphere formation of A549 and CL1-1 lung cancer cells cultured in spheroid culture and also decreased expression of epithelial-to-mesenchymal transition (EMT)-associated proteins and the ability of cells to migrate and invade. A549 and CL1-1 cells were grown in spheroid culture (TS-LC) or adherent culture (CTR) for 12 days. FIG. 11A shows the results of the microarray analysis (comparison of A549 cells cultured in serum or serum-free medium for 12 days). FIG. 11B shows the results of RT-PCR analysis of Col XVII in A549 cells and Western analysis of Col XVII and laminin-5 in A549 and CL1-1 cells. FIG. 11C shows that the knockdown of Col XVII resulted in decreased sphere formation in A549 and CL1-1 cells. FIG. 11D shows that the Western blot analysis showed knockdown of Col XVII downregulated the expression of EMT markers (including Snail and N-cadherin) and upregulated E-cadherin. FIG. 11E shows the results of migration assay after knockdown of Col XVII. FIG. 11F shows the results of invasion assay after knockdown of Col XVII. FIG. 11G shows the results of wound healing assay after knockdown of Col XVII in A549 and CL1-1 cells. The results are expressed as mean±standard deviation of three independent experiments. Asterisks indicate significant differences as determined by student's t-test or one-way ANOVA.

FIGS. 12A-12E show that Knockdown of laminin-5 reduced sphere formation of A549 and CL1-1 lung cancer cells cultured in spheroid culture and also decreased the expression of epithelial-to-mesenchymal transition (EMT)-associated proteins and the ability of cells to migrate and invade. FIG. 12A shows the knockdown of laminin-5 resulted in decreased sphere formation in A549 and CL1-1 cells. FIG. 12B shows the result of Western blot analysis, which showed that knockdown of laminin-5 downregulated the expression of EMT markers (including Snail and N-cadherin) and upregulated E-cadherin. FIG. 12C shows the results of migration assay after knockdown of laminin-5. FIG. 12D shows the results of invasion assay after knockdown of laminin-5. FIG. 12E shows the results of wound healing assay after knockdown of laminin-5 in A549 and CL1-1 cells. The results are expressed as mean±standard deviation of three independent experiments. Asterisks indicate significant differences as determined by student's t-test or one-way ANOVA.

FIGS. 13A-13E show that Collagen XVII shedding stimulated by PMA enhanced laminin-5 expression. Knockdown of ADAMS or ADAM10 inhibited shedding of Collagen XVII and reduced laminin-5 expression in A549 cells cultured in spheroid culture. FIG. 13A shows representative immunoblots of laminin-5, the 180-kDa full-length Collagen XVII (Col XVII 180) and the 120-kDa shed ectodomain of Coll XVII (Col XVII 120, ectoderm) of A549 cell culture when PMA (100 nM) and 1,10-phenanthroline (20 μM) were added respectively in A549 cells cultured in spheroid and cultured for 12 days. FIG. 13B shows results of Western blot analysis when cycloheximide (50 μg/ml) was added to determine if laminin-5 would degrade over time in A549 cells. The results showed decreased degradation of laminin-5 in A549 cells to which PMA was added. FIG. 13C shows the result of Western blot analysis when ADAM9 or ADAM10 was knocked out in A549 cells, which showed increased expression of Col XVII 120 and reduced expression of laminin-5. FIG. 13D shows that when PMA was added, the expression of Snail was enhanced and the epithelial-to-mesenchymal transition (EMT) markers were activated; when 1,10-phenanthroline was added, the expression of Snail and EMT markers was reduced. FIG. 13E shows the results of wound healing ability, cell migration and invasion after the addition of PMA or 1,10-phenanthroline.

FIGS. 14A-14F show that the incidence of lung metastasis was reduced by knockdown of Col XVII or laminin-5 in an animal model. Patients with high expression of Col XVII and laminin-5 had worse prognosis after resection of lung cancer than those with other expression profiles. FIG. 14A shows the results of tail vein injection of A549 lung cancer cells. The group of A549 cells cultured in spheroid culture had evident lung metastasis. The groups of control, knockdown of Col XVII and knockdown of laminin-5 cells had no evidence of lung metastasis 12 weeks after tail vein injection of lung cancer cells. FIG. 14B shows the HE staining of lung sections showed lung metastasis as observed in the animal receiving tail vein injection of A549 cells cultured in spheroid culture. 20×, bars indicate 100 μm; 200×, bars indicate 50 μm. FIG. 14C shows the representative pictures of immunohistochemical stain of Col XVII and laminin-5 in tumor specimens from two patients having pulmonary resection for lung cancer. Bars indicate 50 μm. FIG. 14D-F show the survival curves of patients with positive expression of Col XVII and/or laminin-5 or negative expression of Col XVII and/or laminin-5, which showed that patients with positive expression of Col XVII and laminin-5 had worse prognosis than patients with negative expression of Col XVII or laminin-5 or both.

FIGS. 15A-15E show that knockdown of Col XVII or laminin-5 decreased the expression of phosphorylated FAK, AKT and GSK3b in A549 and CL1-1 lung cancer cells cultured in spheroid culture, indicating that Col XVII and laminin 5 are involved in the FAK/AKT/GSK3β pathway to regulate the epithelial-to-mesenchymal transition (EMT) phenotype. FIG. 15A shows the result of Western blot analysis of phosphorylated FAK, AKT and GSK3β expression in A549 and CL1-1 cells in adherent culture (CTR) or spheroid culture (TS-LC). FIG. 15B shows the result of Western blot analysis of phosphorylated FAK, AKT and GSK3β expression in A549 cells in spheroid culture with Col XVII knockdown. FIG. 15C shows the result of Western blot analysis of phosphorylated FAK, AKT and GSK3β expression in A549 cells in spheroid culture with laminin-5 knockdown. FIG. 15D shows the result of Western blot analysis of phosphorylated FAK, AKT and GSK3β expression when FAK inhibitor (20 μM) and LY 294002 (10 μM) was added. FIG. 15E shows the results of immunoprecipitation assay, which show that the ubiquitination of Snail was suppressed in A549 lung cancer cells cultured in spheroid culture.

FIGS. 16a-g show that spheroid culture-enriched TICs possess hemidesmosome-like plaques and survive under suspension conditions. Aggregate of HT29 bulk cancer cells formed by hanging drop culture (Bulk/Agg) and sphere formed in HT29 spheroid culture (SPH) without or with shRNA against Col XVII (siCol 17a1) and laminin-5 (siLaminin-5) were subjected to immunofluorescence followed by examination with a confocal microscope. CTR was control scrambled shRNA. The results were shown in FIG. 16a. Bar=20 μm. FIG. 16b shows that TEM images show the existence of hemidesmosomes in wild-type HT-29 spheroid culture-formed sphere (arrow), but not in cells with shRNA against Col XVII and laminin-5. Bar=0.2 μm. FIG. 16c-d show respectively the result of TUNEL assay and Western bolt analysis of aggregate of bulk cancer cells formed by hanging drop culture and spheroid culture-enriched TICs cultured in spheroid condition for 24 h. Bar=10 μm. FIG. 16e-f show the result of Live/Dead analysis of cells in FIG. 16a cultured in spheroid condition for 24 h. Bar=20 μm. FIG. 16g shows the result of Live/Dead analysis of spheres formed by spheroid culture treated with trypsin or chymotrypsin treatment for 30 min and subjected to spheroid culture for 24 h. Bar=20 μm.

### DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

The term “inhibiting” refers to the ability of a compound, agent, or method to prevent the onset of the symptoms, alleviating the symptoms, or eliminating the disease, condition or disorder.

As used herein, the terms “prevent”, “preventing”, “prevention” and the like refer to reducing the probability of developing a disorder or condition in a subject, who does not have, but is at risk of or susceptible to developing a disorder or condition.

The term “tumorigenesis” refers to initiation of primary or metastatic tumor growth, and the promotion of invasive growth.

The term “metastasis” as used herein refers to the spread of cancer cells, or cancer stem cells from one organ or part to another non-adjacent organ or part.

The term “cancer stem cells (CSCs)” as used herein refers to cancer cells (found within tumors or hematological cancers) that possess characteristics associated with normal stem cells, specifically the ability to give rise to all cell types found in a particular cancer sample. CSCs are therefore tumorigenic (tumor-forming), at much lower cell numbers than commonly used cell cultures grown in fetal calf serum containing medium. CSCs may generate tumors through the stem cell processes of self-renewal and differentiation into multiple cell types. The terms “tumor stem-like cells” or “tumor initiating cells” are essentially synonymous to the term “cancer stem cells.”

The term “therapeutically effective amount” or “effective amount” refers to a predetermined amount calculated to achieve the desired effect, i.e., to inhibit, block, or reverse the activation, migration, or proliferation of cells.

As used herein, the term “chemical agent” encompasses any chemical molecule or chemical element, or combination of chemical molecules and/or chemical elements. For example, the term “chemical agent” encompasses proteins and peptides.

The term “antibody” relates to derivatives or functional fragments thereof which still retain the binding specificity.

The present invention unexpectedly found that TICs increase phosphorylation of ^{S727}STAT3 by inactivating PP2A, leading to up-regulation of Collagen XVII (Col XVII), which stabilizes laminin 5 to form hemidesmosome-like structures. Targeting PP2A-^{S727}STAT3-Col XVII in TICs blocks their suspension survival and tumour initiation. In contrast, up-regulation of Col XVII, enhancing ^{S727}STAT3 phosphorylation or suppressing PP2A in bulk cancer cells increases their suspension survival and tumour initiation. The ^{S727}STAT3 phosphorylation and Col XVII expression in colorectal cancer samples correlated inversely with patient prognosis and survival. Moreover, targeting TICs through the current pathway in a subcutaneous xenograft model and a malignant pleural effusion model confirms the TIC suspension survival-related pathway is a potential target for curing cancer.

In one aspect, the present invention provides a method for inhibiting and preventing survival, tumorigenesis and metastasis of cancer cells and/or cancer stem cells (CSCs) in a subject comprising administering the subject with a pharmaceutical composition comprising Collagen XVII (Col XVII) inhibitor to CSCs in an amount effective in inhibiting and preventing survival, tumorigenesis and metastasis of said cancer stem cells. In other words, the present invention provides a use of Collagen XVII (Col XVII) inhibitor to cancer stem cells (CSCs) for the manufacture of a medicament for inhibiting and preventing survival, tumorigenesis and metastasis of cancer cells and/or CSCs.

In one embodiment of the invention, the Col XVII is a target of S727-phosphorylated STAT3 (^{S727}STAT3) and responsible for suspension survival and tumor initiation via the activation of the laminin 5-FAK pathway in CSCs.

In the present invention, the Col XVII inhibitor is a component capable of blocking PP2A-^{S727}STAT3-Col XVII pathway, a component capable of inhibiting Col XVII itself, or a component capable of inhibiting ^{S727}STAT3-Col XVII-laminin 5-FAK pathway whereby mediating suspension survival and tumor initiation in CSCs. In one embodiment of the present invention, the Col XVII inhibitor is selected from the group consisting of a microRNA (miRNA), a small interfering RNA (siRNA), a chemical agent, an antibody, an inhibitor of enzyme existing in PP2A-^{S727}STAT3-Col XVII pathway, and an inhibitor of enzyme existing in ^{S727}STAT3-Col XVII-laminin 5-FAK pathway. In one specific embodiment, the chemical agent is PP2A activator or STAT3 inhibitor. In particularly, the PP2A activator is forskolin, 1,9-dideoxy-forskolin, or FTY720, and the STAT3 inhibitor is selected from the group consisting of PY*LKTK, SS 610, 53I-M2001, STA-21, S3I-201, Stattic, IS3 295, CPA-1, CPA-7, Galiellalactone, Peptide aptamers, Decoy ODN, G-quartet ODN, and Peptides. In one another specific embodiment, the antibody is anti-Col XVII monoclonal or polyclonal antibody, or anti-laminin 5 monoclonal or polyclonal antibody.

In another aspect, the present invention also provides a pharmaceutical composition for inhibiting and preventing survival ability, tumorigenesis and metastasis of cancer cells and/or cancer stem cells, comprising an effective amount of Col XVII inhibitor.

In the present invention, the composition in the present invention comprises a Col XVII inhibitor, wherein the Col XVII inhibitor is a component capable of blocking PP2A-^{S727}STAT3-Col XVII pathway, a component capable of inhibiting Col XVII itself, or a component capable of inhibiting ^{S727}STAT3-Col XVII-laminin 5-FAK pathway whereby mediating suspension survival and tumor initiation in CSCs. In one embodiment of the present invention, the Col XVII inhibitor is selected from the group consisting of a microRNA (miRNA), a small interfering RNA (siRNA), a chemical agent, an antibody, an inhibitor of enzyme existing in PP2A-^{S727}STAT3-Col XVII pathway, and an inhibitor of enzyme existing in ^{S727}STAT3-Col XVII-laminin 5-FAK pathway.

In one aspect, the present invention provides use of a CSC surface protein or extracellular protein for the manufacture of a kit for preventing tumorigenicity or treating cancer, wherein the CSC surface protein or extracellular protein is Col XVII.

The present invention is further illustrated by the following examples, which are provided for the purpose of demonstration rather than limitation.

### EXAMPLES

### Example 1. Collagen XVII (Col XVII) Serves as Downstream Target of S727-Phosphorylated STAT3 (^{s727}STAT3) and Mediates Suspension Survival in Cancer Stem Cells

I. Materials and Methods

1. Reagents

Treatment reagents included MG132 (Merk, Schwalbach, Germany), Calyculin A (Cell Signaling Technologies, Beverly, Mass.), Okadaic acid (Merk), Ceramide C6 (Santa Cruz Biotechnology Inc., Santa Cruz, Calif.), Cyclohexamide (Merk), and S3I-201 (Merk).

2. Primary Cells and Cell Lines

All human materials such as fresh human tumour samples or tissue blocks used in this study were approved by Institutional Review Board (IRB) of Taipei Veterans General Hospital (Taipei, Taiwan). The CCS and HCW primary cancer cells, provided by Dr. Wen K. Yang (China Medical University Hospital, Taichung, Taiwan), were isolated from a primary tumour of a 63 y/o female with colorectal adenocarcinoma and from a liver metastasis of a male with colorectal adenocarcinoma, respectively. HT29 (human colorectal cancer cell line) was obtained from the American Type Culture Collection (ATCC). All of CCS, HCW and HT29 cancer cells were grown in DMEM-HG (Gibco, Grand Island, N.Y.) containing 10% fetal bovine serum (FBS; Gibco). A549 (human lung cancer cell line) was obtained from the ATCC and grown in Han's F12 (Gibco) containing 10% FBS. MCF7 (human breast cancer cell line) was obtained from the ATCC and grown in DMEM/F12 (Gibco) containing 10% FBS. HTB186 (human medulloblastoma cell line) was obtained from the ATCC and grown in MEM (Gibco) containing 10% FBS. For spheroid culture, cells were grown in serum-free DMEM-F12 (Gibco) containing 20 ng/mL EGF (Peprotech, Rocky Hill, N.J.), 10 ng/mL bFGF (Peprotech), and N2-supplement (Gibco), with or without PKH26 viable dye labeling (Sigma-Aldrich, St. Louis, Mo.). For PKH26 labeling, cells were labeled with 1:1 mix of 2× Dye for 10 min. For differentiation culture, the spheres after 15 days of spheroid culture were mixed with growth factor-reduced matrigel (Becton Dickinson, San Jose, Calif.) and cultured in growth medium with 15% FBS for one month. Cells were then recovered by commercial solution (Becton Dickinson) for the subsequent experiments.

2. Tissue Collection and Flow Cytometry Cell Sorting

The tissue collection was approved by the Institutional Ethics Committee/Institutional Review Board of the Taipei Veterans General Hospital. Colorectal cancer specimens with disease grade 3 to 4 were collected during surgery and immersed in normal saline, brought to the laboratory within 1 hour, and washed 5 times with phosphate-buffered saline containing 500 U/mL penicillin and 500 g/mL streptomycin (Gibco). Sample were minced into small fragments (2 mm) and resuspended in DMEM-HG (Gibco) containing 1 mg/mL type I Colase (Sigma-Aldrich) for enzymatic dissociation at 37° C. for 2 hour. Released cell samples were subjected to fluorescence-activated cell sorter.

3. Flow Cytometric Analysis and MACS-Separation

Single cell suspension from spheres was incubated with PE-conjugated monoclonal antibodies against human CD133 (MACS; Miltenyi Biotec Ltd., Surrey, UK) for 30 min at 4° C., washed twice with PBS and analyzed with FACScan flow cytometer (Becton Dickinson). The isolation of cells with high Aldehyde Dehydrogenase (ALDH) enzymatic activity was performed by using ALDEFLUOR kit (StemCell Technologies, Durham, N.C.). Briefly, single cells were suspended in ALDEFLUOR assay buffer containing ALDH substrate and then incubated for 30 min at 37° C. without or with a specific ALDH inhibitor, diethylaminobenzaldehyde (DEAB), as a control. Stained cells were sorted on a FACSAria flow cytometer (Becton Dickinson). Magnetic cell separation (MACS) of CD133⁺ cells was performed (Auto-Macs; Miltenyi Biotec Ltd.).

4. Xenograft Transplantation and In Vivo Suspension Experiment

The protocols were approved by the Institutional Animal Committee of Taipei Veterans General Hospital. The athymic nude mice (BALB/cAnN.Cg-Foxnlnu/CrlNarl, National Laboratory Animal Center) were maintained in specific pathogen-free conditions. The mice used for the experiments were 6 to 8 weeks of age. Tumour cells were injected subcutaneously (s.c.) and tumour growth was monitored every week for up to 3 months. For the in vivo suspension experiment, aliquots of 10⁴ cells which delivered in 0.5% methylcellulose without or with matrigel (5%) were loaded to Directed in vivo Angiogenesis Assay (DIVAA) angioreactors (Trevigen, Gaithersburg, Md.) and implanted beneath the skin of nude mice. After 24 hours, cells were recovered from the tubes and processed for TUNEL assay.

5. TUNEL Assay

Detection of apoptosis was performed (Roche Molecular Biochemicals, Indianapolis, Ind.). Cells were fixed with 4% paraformaldehyde, and rinsed with PBS, then incubated with blocking solution (3% H₂O₂ in methanol) for 10 min. The cells were rinsed with PBS, and permeabilized by 0.1% Triton X-100 in 0.1% sodium citrate for 2 min at 4° C., then incubated with reaction mixture for 60 min at 37° C. in the dark. The mixture was rinsed with PBS, and counterstained with DAPI. Immunofluorescence is observed with fluorescence microscope.

6. Quantitative Real-Time PCR (Quantitative RT-PCR)

TRIzol reagent (Invitrogen, Carlsbad, Calif.) was employed to extract total RNA, and RNA was reversely transcribed using Superscript III RT Kit (Invitrogen). The quantitative real-time PCR was performed using FastStart SYBR Green Master (Roche Applied Science, Mannheim, Germany) and ABI Step One Real-Time PCR System machine. The sequences of primer sets are listed in Table 1.

TABLE 1
Primer sequences for real-time PCR

| Primer name | Primer sequences |
|---|---|
| Col17A1 F | 5′-AGGCCAGAGCAAACAGAAAA-3′ |
| Col17A1 R | 5′-ATGGAGGGTGACGTCTTGAG-3′ |
| SPRR1A F | 5′-GACCACACAGCCCATTCTG-3′ |
| SPRR1A R | 5′-TAGAGGTGCAAAGGAGCGAT-3′ |
| SPRR1B F | 5′-ACTGTTGCAGCATGAGTTCC-3′ |
| SPRR1B R | 5′-CTCCTTGGTTTTGGGGATG-3′ |
| ECM1 F | 5′-AGCAGCTGGGACTGAGTCAT-3′ |
| ECM1 R | 5′-AAGCTTGTCTGGTGGCTGTT-3′ |
| MXD1 F | 5′-GTGCCTGGAGAAGTTGAAGG-3′ |
| MXD1 R | 5′-CTGAAGCTGGTCGATTTGGT-3′ |
| RAB3B F | 5′-GAGAGGGTTGTTCCCACTGA-3′ |
| RAB3B R | 5′-AAAGGCCTGCCTTACACTGA-3′ |
| BHLHE40 F | 5′-GATCCTGCTGCTTTGCTTTC-3′ |
| BHLHE40 R | 5′-CACACACACACACACCCTGA-3′ |
| CD55 F | 5′-TTCACCATGATTGGAGAGCA-3′ |
| CD55 R | 5′-CTGAACTGTTGGTGGGACCT-3′ |
| SAV1 F | 5′-CTGTCCCGAAAGAAAACCAA-3′ |
| SAV1 R | 5′-GGCATAAGATTCCGAAGCAG-3′ |
| Laminin V F | 5′-GGCTGGTCTTACTGGAGCAG-3′ |
| Laminin V R | 5′-CATCAGCCAGAATCCCATCT-3′ |
| GAPDH F | 5′-CTCTGCTCCTCCTGTTCGACA-3′ |
| GAPDH R | 5′-ACGACCAAATCCGTTGACTC-3′ |

7. Western Blot Analysis

Cells were lysed and protein was extracted using M-PER (Pierce, Rockford, Ill.) plus protease inhibitor cocktail (Pierce), and the protein concentrations were determined by the BCA assay (Pierce). Aliquots of protein lysates were separated on SDS-10% polyacrylamide gels and transferred onto poly-vinylidenedifluoride (PVDF) membrane, which was blocked with 5% milk (Bio-Rad, Richmond, Calif.) in TBST. The membrane was then hybridized with primary antibodies followed by corresponding secondary antibodies, and then detected using a chemiluminescence assay (Millipore, Billerica, Mass.). Membranes were exposed to X-ray film to visualize the bands (Amersham Pharmacia Biotech, Piscataway, N.J.). Antibodies against pSTAT3 (S727) (1:1000), pSTAT3 (Y705) (1:1000), STAT3 (1:2000), PP2A C subunit (1:2000), pAkt (S473) (1:1000), β-tubulin (1:2000), Flag (1:2000), pAkt (T308) (1:1000), Akt (1:2000), ERK (1:2000), pP38 (T180 and Y182) (1:1000), P38 (1:2000), pJNK (1:1000), and pmTOR (S2448) (1:1000) were purchased from Cell Signaling Technologies. Antibodies against pPP2A (Y307) (1:2000), Oct4 (1:500), Nanog (1:500), Histone H1 (1:1000), and Sox2 (1:500) were purchased from Santa Cruz Biotechnology Inc. Antibodies against pFAK (Y397) (1:1000) were purchased from GeneTex (San Antonio, Tex.). Antibody against Col17a1 was purchased from Abcam. Antibody against Laminin y2 (1:1000) was purchased from Chemicon (Temecula, Calif.). Antibody against cleaved-caspase 3 (1:1000) was purchased from Epitomics (Burlingame, Calif.). Antibody against pERK (1:1000) was purchased from Biosource (Camerillo, Calif.).

8. Immunoprecipitation

Cell extracts were incubated with antibodies against PP2A (Cell Signaling Technologies) and STAT3 (Cell Signaling Technologies) overnight at 4° C. with gentle rotation. The immune complexes were collected by adding protein G beads (Millipore) and incubated for 1 hour at 4° C. with gentle rotation followed by centrifugation. Precipitates were washed with ice-cold PBS. The precipitates were suspended in SDS sample buffer and analyzed by SDS-polyacrylamide gel. Immunoblotting was performed by antibodies against PP2A, and STAT3.

9. Immunofluorescence

Cells were fixed with 4% paraformaldehyde, and reacted with primary antibodies against human CDX2 (Chemicon) (1:200), pSTAT3 (S727) (1:10), cleaved-caspase3 (1:100), β-tubulin (1:10), Col17a1 (1:50), Laminin y2 (1:50), and pPP2A (Y307) (1:50). The cells were washed with PBS containing 0.1% Triton X-100, reacted with corresponding DyLight™ 488, DyLight™649 or DyLight™594-conjugated secondary antibodies (Jackson ImmunoResearch Laboratories, West Grove, Pa.), and counterstained with DAPI. Immunofluorescence was observed with a fluorescence microscope or a confocal fluorescence microscope.

10. Tissue Microarray Slides and Immunohistochemistry

The use of human tissues or tumour specimen samples was approved by the Institutional Review Board of Taipei Veterans General Hospital (Taipei, Taiwan). For immunohistochemical staining, paraffin-embedded sections were deparaffinized and rehydrated, with antigen retrieved by placing sections in Declere working solution (Cell Marque, Austin, Tex.) in 95° C. water for 30 min. Endogenous peroxidase activity was blocked by 3% hydrogen peroxide. Residual enzymatic activity was removed by washes in PBS, and non-specific staining was blocked with Ultra V Block for 5 min (Thermo Fisher Scientific, Fremont, Calif.). Then the sections were reacted with primary antibodies: pS727-STAT3 (1:25) and Col17a1 (1:50) followed by corresponding biotinylated secondary antibodies (Vector Laboratories, Burlingame, Calif.), treated with streptavidin-peroxidase (LSAB Kit; Dako, Carpinteria, Calif.), and followed by diaminobenzidine staining. Counterstaining was performed with Mayer's hematoxylin and photographed with Zeiss Axiolmager Z1 microscope system (Wetzlar, Germany) and an automated acquisition system (TissueGnostics, Vienna, Austria). Pictures were acquired using Tissue-Faxs software (TissueGnostics). The percentage of positively stained cells was determined using HistoQuest software (TissueGnostics).

11. Plasmid Reconstruction

The pHM6-CMV-PP2A (WT and H59K) plasmids were provided by Dr. CW Chiang in Chang-Gung University (Taipei, Taiwan). The pHM6-CMV-PP2A (WT and H59K) plasmids were subcloned into PCDNA3 plasmids. The pXJ40-CMV-STAT3 (S727A and Y705F) plasmids were obtained from Dr. Steven Plelech in Brain Research Center and Department of Medicine, University of British Columbia. The lentiviral-based expression plasmids for S727A (SA), S727E (SE), and Y705F+S727E (YFSE) point-mutated STAT3 were generated by subcloning pXJ40-CMV-STAT3 (S727A and Y705F) plasmid into pLKO-AS2.puro plasmids, followed by site-directed mutagenesis PCR.

12. Lentiviral Vector Production and Cell Infection

The shRNA expression plasmids and the bacteria clones for STAT3 (TRCN-00000020840, TRCN-0000020843) or Col17a1 (TRCN-0000118937, TRCN-0000118940) were provided by the RNAi core of the National Science Council in Taiwan. Lentiviral production for shRNA and point-mutated STAT3 over-expression were performed by transfection of 293T cells using Lipofectamine 2000 (LF2000; Invitrogen, Carlsbad, Calif.). Cells were infected in the presence of 8 μg/mL polybrene (Sigma-Aldrich) and selected with puromycin (1 g/mL).

13. Anchorage-Independent Growth Assay

Agarose culture medium (1%) containing 10% FBS was employed to coat the bottom of culture plates. After hardening, 500, 1000, and 2500 cells were suspended in agarose culture medium (0.8%) containing 10% FBS and plated onto the bottom layer. Colonies formed in the soft agarose culture were stained with crystal violet and counted 2 weeks after inoculation.

14. Gene Expression Profiling

Total RNA was isolated using the Qiagen RNeasy kit. The generation of labeled cRNA and its hybridization to Human U133 Plus 2 GeneChip arrays (Affymetrix) were done at the genomic core facilities in National Yang-Ming University (Taipei, Taiwan). Functional annotation and identification of overrepresented functional themes were done using Ingenuity Pathway Analysis (IPA) web tool developed by Ingenuity Co. All data were deposited in the Gene Expression Omnibus (GEO) database with an accession number of GSE43576.

15. ChIP Assay

Chromatin immunoprecipitation (ChIP) assay was performed by a commercial kit (Upstate Biotechnology). HT29 cells with stably overexpressed S727E-mutated STAT3 were fixed with 1% formaldehyde for 20 min at 37° C., and lysed on ice for 10 min in lysis buffer. DNA-protein complexes were sonicated to 200 and 600 base pairs. One aliquot of chromatin was stored for use as input DNA, and the remainder was diluted in immunoprecipitation (IP) buffer, and incubated overnight (4° C.) with anti-Flag antibody (Sigma-Aldrich). DNA-protein complexes were isolated on salmon sperm DNA/protein A agarose beads and then eluted. Cross-linking was reversed by incubation at 65° C. for 4 hours. Proteins were removed with proteinase K. DNA was extracted with phenol/chloroform, and PCR-amplified with specific primer for conserved STAT3 binding sequence.

16. Luciferase Reporter Assay

Luciferase assay was performed by the Secrete-Pair Dual Luminescence Assay kit (GeneCopoeia, Rockville, Md.). All transfections were performed by Nucleofector technology (AMAXA Biosystems, Cologne, Germany). Wild-type and mutated Col17a1 promoters were cloned in pEZX-PG04 reporter plasmid (GeneCopoeia).

17. Ultrastructural Analysis

Spheres were fixed in 2.5% glutaraldehyde (phosphate buffer) overnight at 4° C. and postfixed in 2% osmium tetroxide (Millonigs buffer) for 90 min at 4° C. after washing with phosphate buffer. Specimens were dehydrated through a graded series of ethanol and embedded in Epon 812-equivalent (TAAB Lab). Semi-thin sections (1 nm) were stained with toluidine blue for light microscopy analysis. Ultra-thin sections (40-90 nm) were cut, stained with uranyl acetate and lead citrate and examined with a Hitachi H7600 transmission electron microscope. For pretreatment of spheres with enzymes, spheres were incubated with chymotrypsin (25 ng/μl) and trypsin (25 ng/μl) in medium for 5 min, followed by washing with PBS twice and subject to suspension culture for 24 hours.

18. Isolation of Cancer Cells from Malignant Pleural Effusion

Malignant pleural effusion obtained aseptically in heparinized (10 U/ml) bottles were centrifuged at 200 g for 20 min at room temperature and cell pellet was resuspended in 30 ml of cold 1% BSA/2 mM EDTA/PBS. After counting by Trypan Blue exclusion method, cell suspension was layered on Ficoll gradient solution and centrifuged at 800 g for 30 min at room temperature. After centrifugation, cells contained in the upper gradient were washed once with 1% BSA/2 mM EDTA/PBS and plated in spheroid medium at a concentration of 10⁵ cells/ml in ultralow attachment plates (Corning). Half volume of spheroid medium was added every 3 days.

19. Mouse Model of Malignant Pleural Effusion

Animal model of malignant pleural effusion was developed by using male athymic nude mice according to protocols modified from Stathopoulos et al (Stathopoulos et al., Current opinion in pulmonary medicine 15, 343-352, 2009). In brief, the animals were anesthetized with 2% Rompun (Bayer Pharma, Puteaux, France) at 5 mg/kg and Zoletil 100 (VirbacR, Carros, France) at 30 mg/kg, and administered intraperitoneally (i.p.). Tumour cells were implanted through the chest wall into the left pleural space of mice (i.p.) in a volume of 200 μl using a 26 gauge needle. The depth of needle penetration through the intercostal muscles was controlled to avoid lung injury and hemorrhage into the pleural space. Prior to being returned to their cages, mice were placed until recovery under a heat lamp to maintain body temperature.

Statistical Analysis

Comparisons between two groups were analyzed by Student's t-test. Comparisons within three groups were analyzed by ANOVA test. Comparison of patient survival curve was analyzed by Log-rank test. Comparison of in vivo tumourigeneicity was analyzed by Fisher's exact test. A value of P<0.05 was considered statistically significant.

II. Results

1. TICs show suspension survival ability

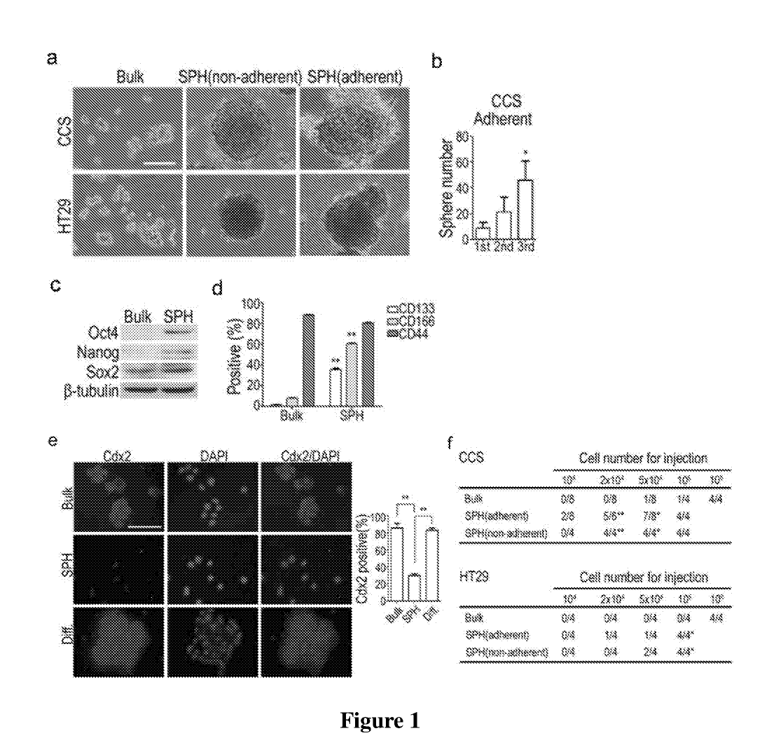

To avoid the unreliability of the isolation TICs by putative surface markers and the selection of cells preferred to survive suspension in ultralow dishes, TICs were enriched by the spheroid culture both in ultralow (FIG. 1a) and ordinary plastic dishes (FIG. 1a) from primary cancer cells, CCS, or cell line, HT29. Enriched TICs increased in the ability to form secondary and tertiary spheres (FIG. 1b), expressed pluripotency factors (FIG. 1c), increased in TIC surface makers, such as CD133 and CD166 (FIG. 1d), decreased in colorectal differentiation marker, such as Cdx2 (FIG. 1e), differentiated when reseeded with serum in matrigel (FIG. 1e), and generated tumours upon inoculation underneath the skin of immunodeficient mice (FIG. 1f).

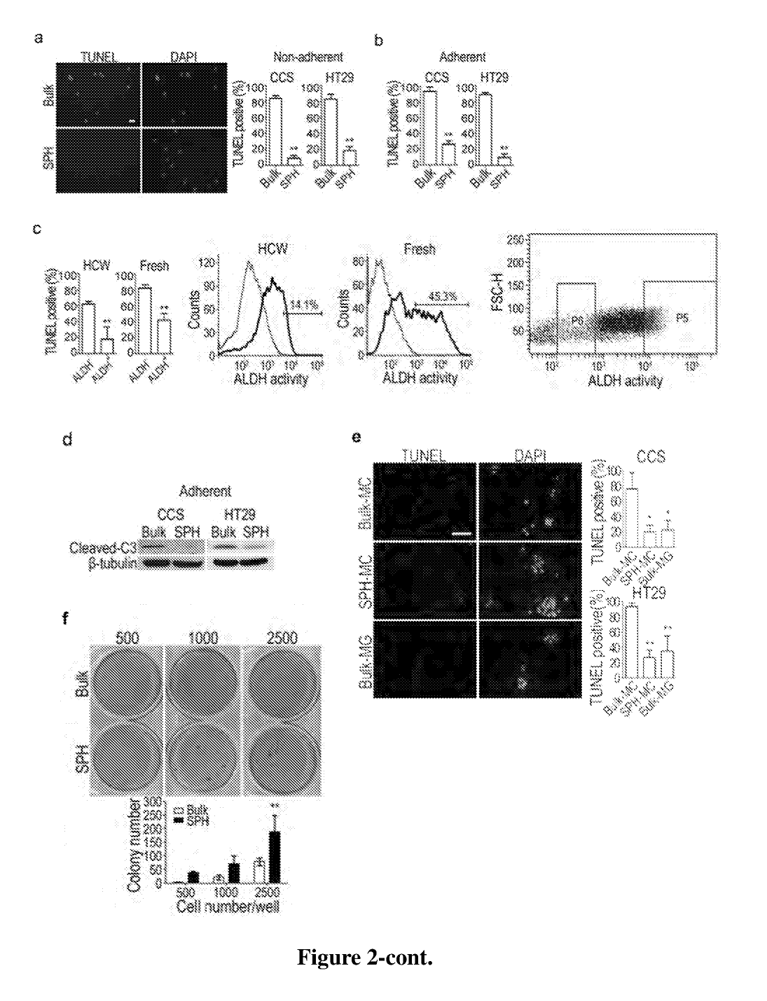

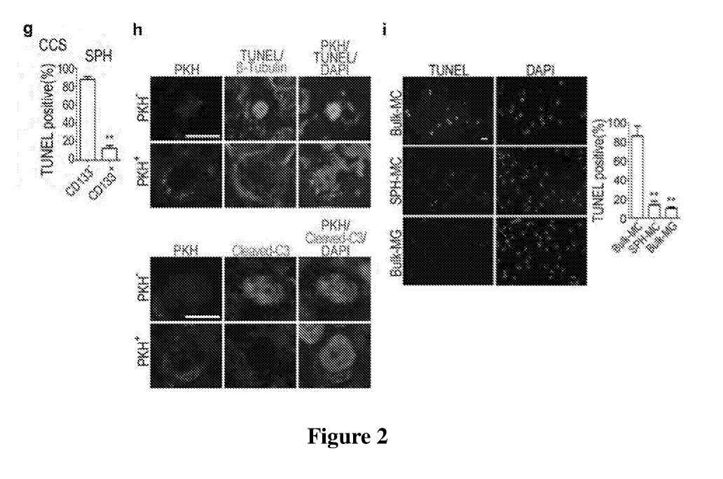

The enriched TICs of CCS and HT29 showed less apoptosis than their bulk cancer cells at 24 hours of suspension with serum deprivation (FIGS. 2a and 2b), independent of culture dishes used to enrich TICs. Moreover, TICs selected by using positive ALDH activity from primary liver metastasis of colorectal cancer cells HCW and fresh colorectal cancer specimens also showed superior suspension survival than ALDH⁻ cancer cells (FIG. 2c). Consistent with previous findings, our results also showed that activation of caspase 3 played a major role in mediating suspension-induced cell death (FIG. 2d). Increased suspension survival by TICs was also demonstrated in condition with serum using poly-HEMA-coated dishes (FIG. 2e) and in long-term soft-agar culture (FIG. 2f). Moreover, the increase of suspension survival in TICs from CCS was attributed mainly to CD133⁺, rather than CD133⁻ cells (FIG. 2g). When compared the survival of TICs and bulk cancer cells using the mitotic-quiescence properties of TICs to retain specific dyes, such as PKH26, in culture, PKH-positive cells survived in spheres compared with PKH-negative cells at 15 days of CCS spheroid culture, which showed positive for TUNEL and cleaved-caspase 3 (FIG. 2h).

To demonstrate that TICs also increased suspension survival in vivo, the nude mice subcutaneously implanted with silica tubes containing methylcellulose-delivered cells. Transplants recovered 24 hours later revealed survival of the majority of enriched TICs from CCS, while bulk cancer cells underwent apoptosis, which can be prevented by adding ECM in methylcellulose (FIG. 2i). These data together suggest that TICs display better suspension survival in a variety of conditions, both in vitro and in vivo.

2. Phosphorylation of STAT3 at S727 Mediates Suspension Survival in TICs

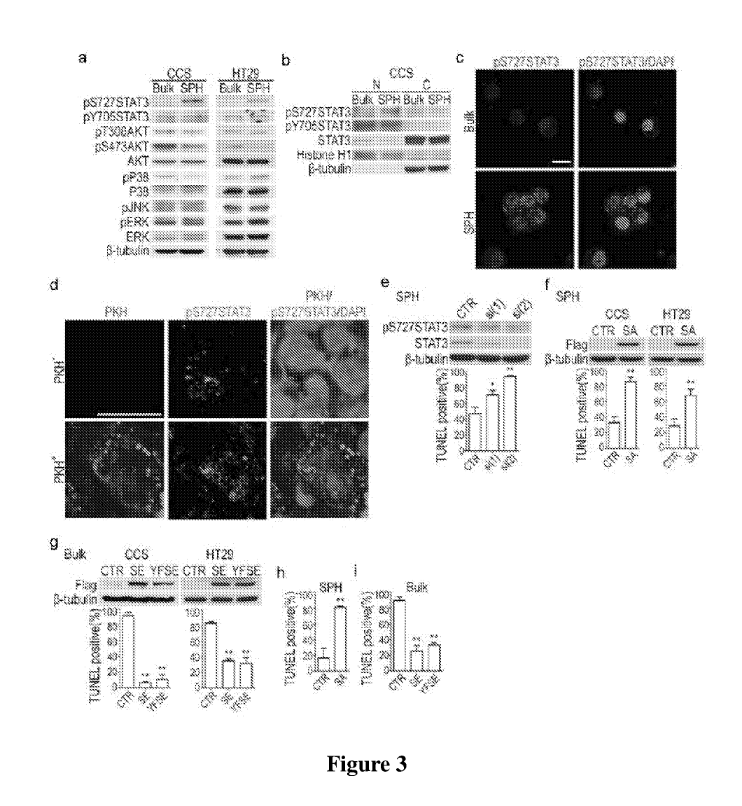

Anoikis is the apoptosis induced by loss of cell adhesion, and is associated with the state of cell differentiation. Immunoblotting analysis revealed no differences in the activation of anoikis-related pathways between TICs and bulk cancer cells under suspension (FIG. 3a). The activation of JAK2-^{Y705}STAT3 pathway by IL-6 has been demonstrated that leads to increased tumour initiation ability in colorectal, lung and breast TICs. Phosphorylation of STAT3 was increased at S727 in TICs compared with bulk cancer cells, while there was no difference between phosphorylation at Y705 and several target genes of Y705-phosphorylated STAT3, including MCL-1, cyclin-D and c-Myc (data not shown), between TICs and bulk cancer cells (FIG. 3a). Moreover, phosphorylated S727 was located in the nucleus, where STAT3 transcription factor functions (FIGS. 3b and 3c). Consistently, S727 phosphorylation was also increased in PKH-positive cells under long-term spheroid culture (FIG. 3d). Moreover, STAT3 knockdown (FIG. 3e) or overexpression of S727A point-mutated STAT3, an inactive form, increased apoptosis in TICs (FIG. 3f), while overexpression of S727E point-mutated STAT3, an active form, decreased apoptosis in bulk cancer cells (FIG. 3g). Similar results were also found in in vivo studies (FIGS. 3h and 3i). More importantly, phosphorylation of STAT3 at Y705 was dispensable for the inhibition of apoptosis in bulk cancer cells by overexpression with S727E point-mutated STAT3 (FIGS. 3g and 3i), suggesting phosphorylation of ^{S727}STAT3 mediates suspension survival in TICs.

3. Col XVII serves as downstream target of S727-phosphorylated STAT3 and mediates suspension survival in TICs

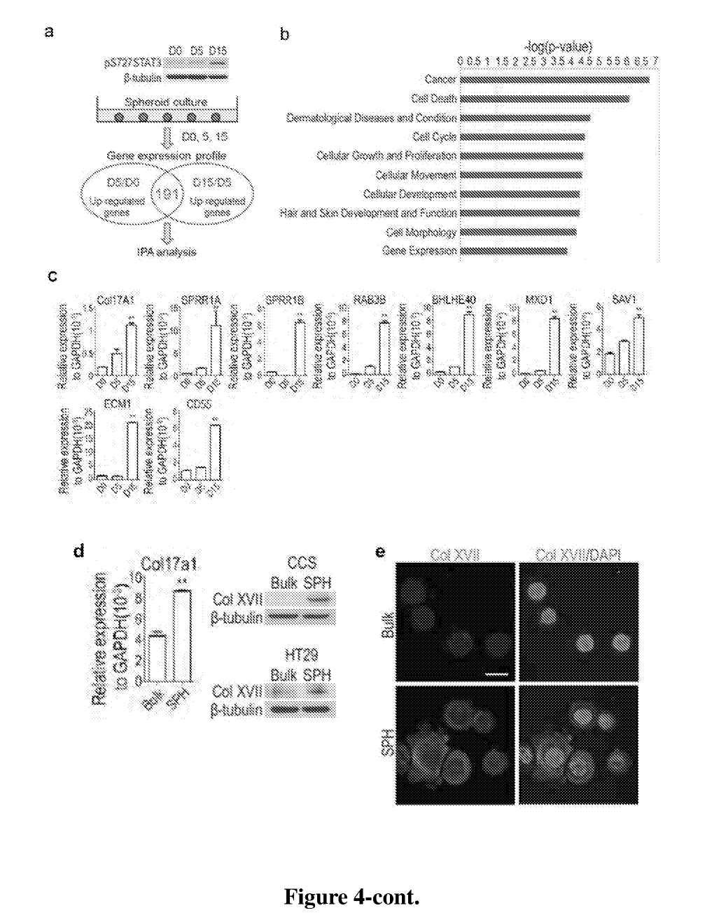

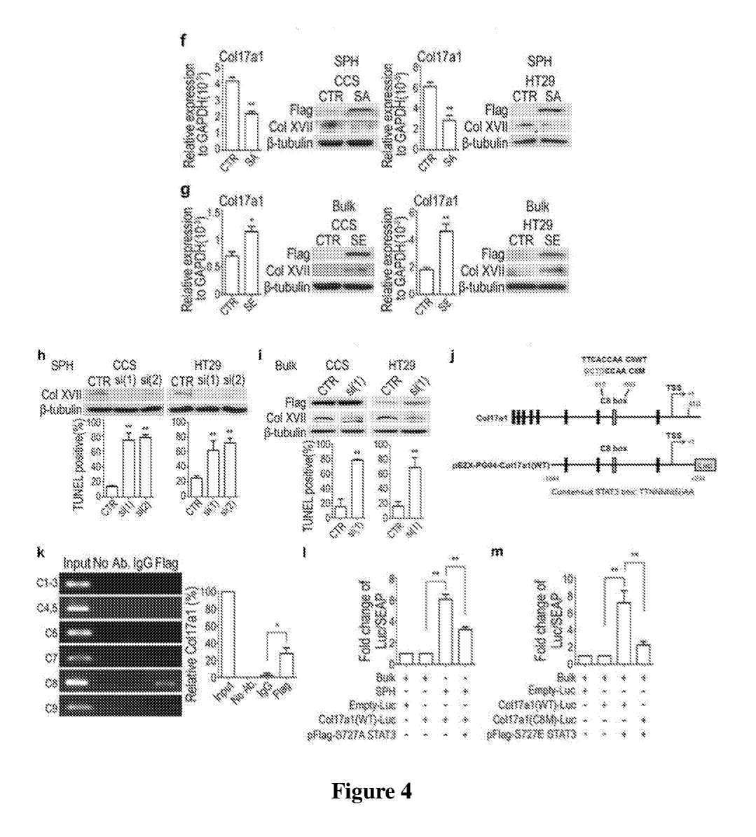

Through Gene Ontology analysis of genes up-regulated along with the increase of spheroid culture time (FIG. 4a), we observed that the genes involved in spheroid culture were specifically enriched in the highly expressed categories, such as cancer and cell death categories (FIG. 4b). Surprisingly, the most up-regulated gene in spheroid culture was Col17a1 (Table 2), which has not been reported to be involved in tumourigenicity and survival ability of cancer cells. The present invention firstly confirmed reliability of the microarray data (Table 2, FIG. 4c) and the up-regulation of Col17a1 during spheroid culture using quantitative RT-PCR, immunoblotting and immunofluorescence (FIGS. 4d and 4e). We then demonstrated that overexpression of S727A point-mutated STAT3 reduced Col17a1 expression in TICs (FIG. 4f), while overexpression of S727E point-mutated STAT3 increased Col17a1 expression in bulk cancer cells (FIG. 4g). Most interestingly, knockdown of Col17a1 reduced suspension survival in enriched TICs (FIG. 4h) and in bulk cancer cells expressing S727E point-mutated STAT3 (FIG. 4i).

Chromatin immunoprecipitation (ChIP) assay of the Col17a1 promoter (FIG. 4j) revealed that fragments containing the 8th putative STAT3 binding sites, TTNNNN(N)AA (−610˜−603), but not other binding sites, were immunoprecipitated with anti-Flag antibody in HT29 expressing S727E point-mutated STAT3 (FIG. 4k). Moreover, the promoter luciferase reporter assay showed that Col17a1 promoter activity was greater in enriched TICs than in bulk cancer cells (FIG. 4l). We further demonstrated that transfection of S727A point-mutated STAT3 inactivated the Col17a1 promoter in enriched TICs (FIG. 4l), and transfection of S727E point-mutated STAT3 activated the wild-type Col17a1 promoter activity but not the 8th binding site-mutated Col17a1 promoter in bulk cancer cells (FIG. 4m). These data together suggest that Col17a1 plays an essential role in suspension survival mediated by S727-activated STAT3 in TICs.

TABLE 2
Up-regulated gene expression along with the increase of spheroid culture time

| Symbol | Entrez Gene Name | D 5/D 0 | D 15/D 5 | D 15/D 0(Log2 Ratio) |
|---|---|---|---|---|
| COL17A1 | collegen, type XVII, alpha 1 | 3.5 | 2.7 | 5.5 |
| KRT6B | keratin 6B | 3.2 | 1.7 | 4.7 |
| CD47 | CD47 molecule | 4.3 | 0.8 | 4.5 |
| KRT15 | keratin 15 | 3.3 | 1.6 | 4.5 |
| KRT6A | keratin 6A | 2.6 | 1.9 | 4.6 |
| SPRR1B | small proline-rich protein 1B | 0.6 | 3.3 | 4.4 |
| SPRR1A | small proline-rich protein 1A | 1.5 | 2.7 | 4.1 |
| DHRS9 | dehrogenase/reductase (SDR family) member 9 | 1.4 | 2.7 | 4 |
| ECM1 | extracellular matrix protein 1 | 0.7 | 3.8 | 3.6 |
| MXD1 | MAX dimerization protein 1 | 1.6 | 1.9 | 3.2 |
| CEACAM1 | carcinoembryonic antigen-related cell adhesion molecule 1 | 2.2 | 0.9 | 3.1 |
| HYAL1 | hyaluronoglucosaminidase 1 | 1.6 | 1.1 | 3.1 |
| RAB3B | RAB3B. member RAS oncogene family | 1.9 | 0.9 | 2.9 |
| AQP3 | aquaporin 3 (Gill blood group) | 1.9 | 0.9 | 2.7 |
| CD55 | CD55 molecule, decay accelerating factor for complement | 1.4 | 1.6 | 2.7 |
| SAV1 | salvador homolog 1 (Drosophila) | 0.8 | 2.0 | 2.7 |
| CYP3A5 | cytochrome P450, family 3, subfamily A, polypeptide 5 | 0.7 | 1.8 | 2.5 |
| LMO7 | LIM domain 7 | 1 | 1.3 | 2.4 |
| STARD4 | StAR-related lipid transfer (START) domain containing 4 | 1.1 | 1.3 | 2.4 |
| TMPRSS4 | transmembrane protease. senne 4 | 1.2 | 1.2 | 2.4 |
| BHLHE40 | basic helix-loop-helix family, member e40 | 0.7 | 1.5 | 2.3 |
| SLC5A3 | solute carrier family 5 (sodium/myo-inositol cotransporter) | 1 | 1.7 | 2.3 |
| LUM | lumican | 1.5 | 1.4 | 2.2 |
| RASSF5 | Ras association (RalGDS/AF-8) domain family member 6 | 1.2 | 1.2 | 2.2 |

4. Suspension survival mediated by Col XVII depends on laminin 5

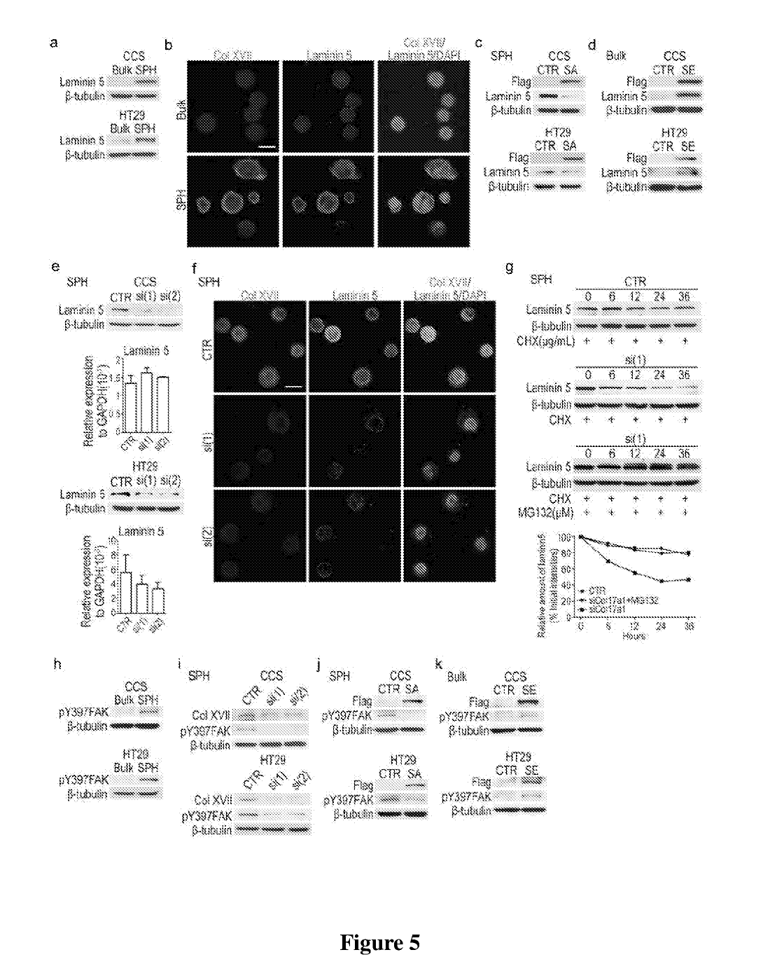

Previous report demonstrates that Col XVII and laminin 5 are components of hemidesmosome for mediating cell adhesion. Moreover, laminin 5 was reported to regulate anchorage-independent survival through downstream signaling, such as FAK. The present invention showed the increase in protein level of laminin 5 in TICs but not in bulk cancer cells (FIG. 5a) and the co-localization of Col XVII and laminin 5 in the membrane and cytoplasm of TICs (FIG. 5b). The present invention further showed the reduction of laminin 5 protein level in spheroid culture of cells that were over-expressed with S727A point-mutated STAT3 (FIG. 5c), but increment of laminin 5 protein level in bulk cancer cells that were over-expressed with S727E point-mutated STAT3 (FIG. 5d). Moreover, knockdown of Col17a1 in TICs reduced the protein level but not the mRNA level of laminin 5 (FIGS. 5e and 5f). Interestingly, the decrease in laminin 5 protein level was due to proteasome degradation, which was restored by treatment with proteasome inhibitor, MG132 (FIG. 5g). Moreover, FAK, a downstream mediator of laminin 5, was also activated in TICs (FIG. 5h) but not in TICs with Col17a1 knockdown (FIG. 5i) or expressing S727A point-mutated STAT3 (FIG. 5j). As expected, FAK was also activated in bulk cells expressing S727E point-mutated STAT3 (FIG. 5k).

These data suggest that Col XVII mediates suspension survival through maintaining the stability of laminin 5.

5. Col XVII and laminin 5 form organized structure to support suspension survival

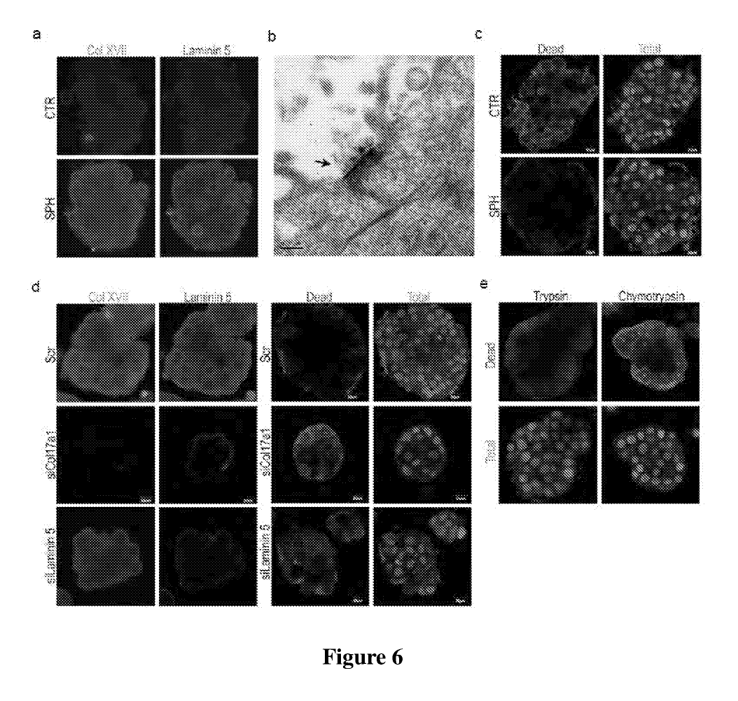

To exclude the possibility that suspension survival by TICs in spheres was mediated by merely cell aggregation, we demonstrated the expression of Col XVII and laminin 5 and the existence of hemidesmosome-like plaques, an organized ECM ultrastructure in the basal lamina of skin epidermis and normal mucosa, in spheres but not in the aggregates of bulk cancer cells formed by hanging drop culture (FIGS. 6a and 6b). When subject to suspension culture for 24 hours, live/dead analysis revealed all cells in spheres were live, while only a small number of cells in the center of the aggregates of bulk cancer cells were live (FIG. 6c). Cells with Col17a1 or laminin 5 knockdown decreased in the ability to form spheres and most of the cells in spheres were dead when subjected to suspension culture (FIG. 6d). Moreover, pre-treatment of the spheres with enzymes that degrade the components of hemidesmosome, such as chymotrypsin, but not trypsin, an enzyme that does not degrade hemidesmosome, for 5 min, cause cell death in suspension culture (FIG. 6e). Together, these data suggest Col XVII and laminin 5 form organized structure in spheres, which plays an important role in supporting suspension survival of TICs.

6. PP2A serves as upstream regulator of STAT3 activation at S727 and mediates suspension survival in TICs

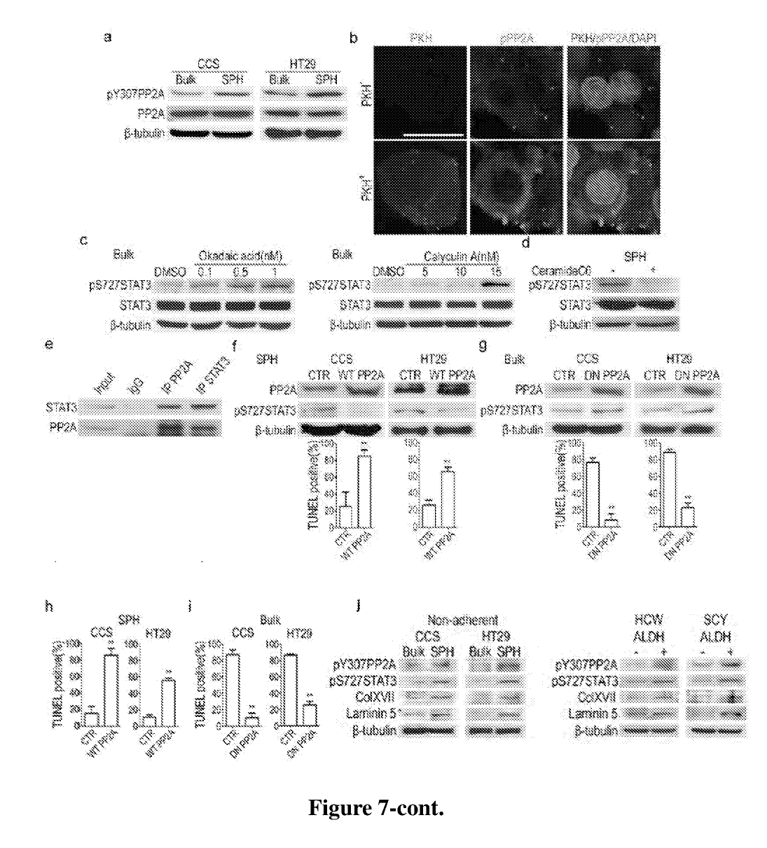

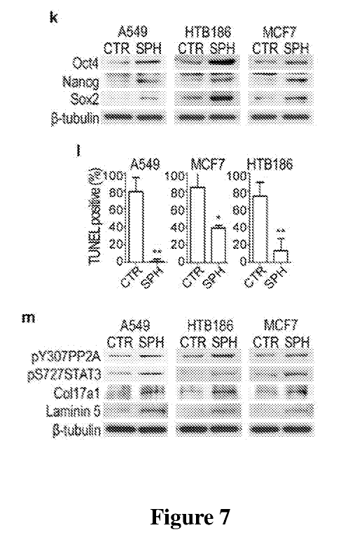

PP2A phosphatase has been reported to inactivate STAT3 via dephosphorylating S727. The phosphorylation of ^{Y307}PP2A, which was associated with suppressed PP2A activity, was higher in both enriched TICs (FIG. 7a) and PKH-positive cells in spheroid culture (FIG. 7b). Treatment of bulk cancer cells with PP2A inhibitors, okadaic acid (OA) and calyculin A (CA) increased S727 phosphorylation of STAT3 in a dose-dependent manner (FIG. 7c). In contrast, treatment of enriched TICs with PP2A activator, ceramide C6, reduced S727 phosphorylation of STAT3 (FIG. 7d). Immunoprecipitation assay further showed that PP2A formed a complex with STAT3 (FIG. 7e), suggesting regulation of STAT3 phosphorylation in TICs by PP2A through a direct interaction. As expected, suspension survival of TICs over-expressing wild-type (WT) PP2A was obviously reduced both in vitro and in vivo (FIGS. 7f, 7h). In contrast, bulk cancer cells expressing dominant negative (DN) form of PP2A increased in suspension survival both in vitro and in vivo (FIGS. 7g, 7i). Consistently, the increase of PP2A-S727STAT3-Col XVII pathway was also demonstrated in TICs enriched by spheroid cultures and ALDH⁺ cells isolated from HCW primary liver metastasis cancer cells, and fresh colorectal cancer specimens (FIG. 7j).

TICs enriched by spheroid culture from A549 lung cancer cells, HTB186 brain cancer cells, and MCF7 breast cancer cells showed increased expression of Oct4, Nanog and Sox2 (FIG. 7k), and increased suspension survival compared with bulk cancer cells (FIG. 7l). Moreover, these cells increased in the phosphorylation of PP2A, and subsequent activation of STAT3 at S727 and the expression of Col17a1 (FIG. 7m), suggesting the suspension survival pathway is a general characteristic in enriched TICs derived from a variety of tumours.

7. Suspension survival in TICs determines tumour initiation ability and corresponds to clinical tumour staging and survival

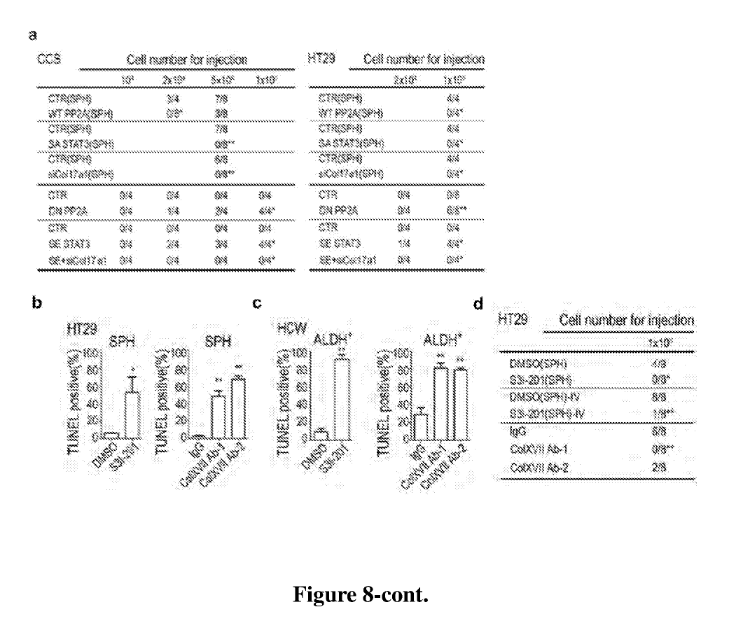

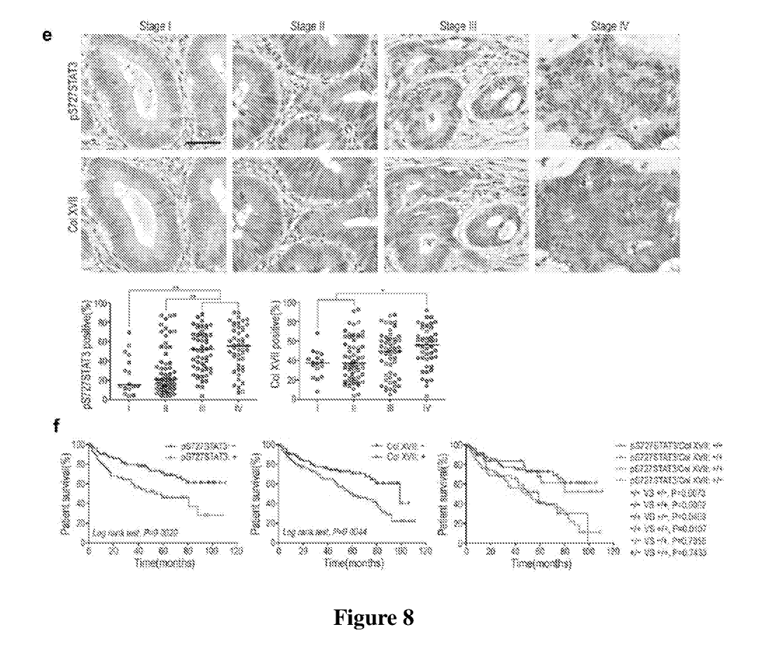

To demonstrate the increased suspension survival ability is essential for tumour formation of TICs and has clinical relevance, the present invention firstly showed that blocking the PP2A-^{S727}STAT3-Col XVII pathway inhibited tumour initiation by TICs (FIG. 8a) and activation of the pathway enhanced tumour initiation in bulk tumour cells to the same extent as in TICs (FIG. 8a). We also showed that targeting STAT3 by using specific inhibitor, S3I-201, killed TICs in suspension culture (FIGS. 8b and 8c) and inhibited tumour initiation (FIG. 8d). Interestingly, targeting Col XVII using anti-Col XVII antibody also killed TICs in suspension culture (FIGS. 8b and 8c) and inhibited tumour initiation (FIG. 8d).

We then examined specimens from 150 colorectal cancer patients and showed increase in phosphorylation at S727 of STAT3 and Col17a1 expression in correspondence with tumour stages (FIG. 8e) and the expression of these two markers inversely correlated with patient survival (FIG. 8f). Upon performance of multivariate survival analysis, the expression of these two markers inversely correlated with patient survival in stage II+III (p=0.046). These data suggest that suspension survival pathway in TICs has clinical relevance and may be used to develop new strategy in targeting TICs.

8. Increase in suspension survival and related pathway in TICs of malignant pleural effusion

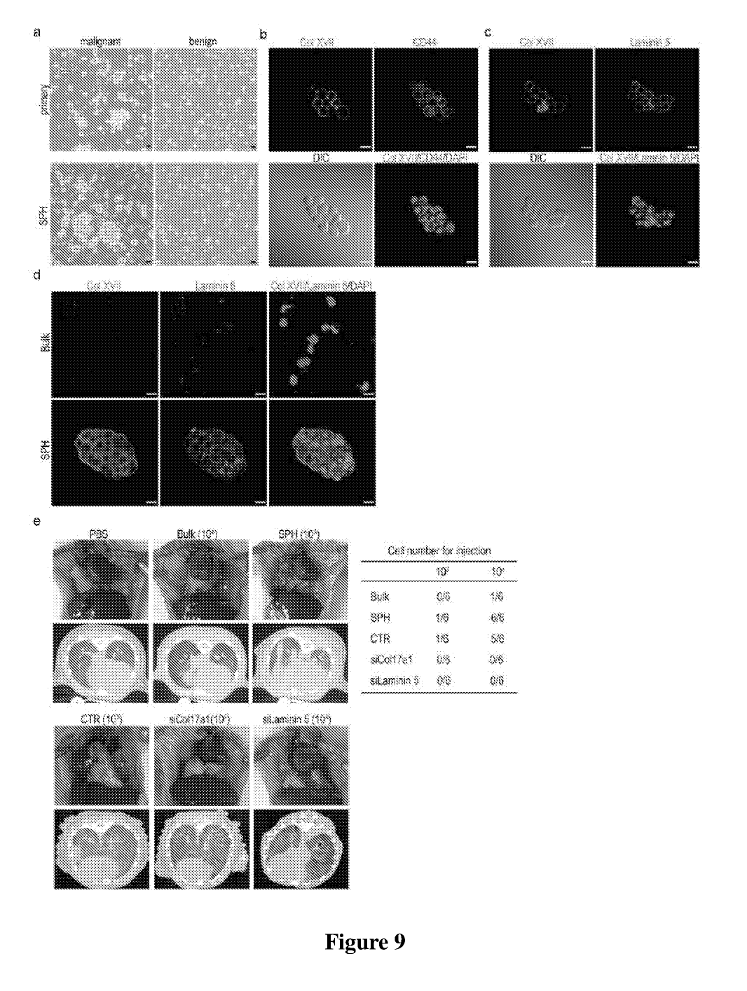

Malignant pleural effusion of lung cancer is a genuine suspension condition in patients' bodies and contains CD44⁺ TICs. After isolation of cancer cells from cytology-confirmed malignant pleural effusion by use of density gradient centrifugation, viable CD44⁺ cells clustering as spheres were observed and could be maintained in spheroid culture (FIGS. 9a and 9b). However, these clustering cells were not observed in benign pleural effusion (FIG. 9a). Immunofluorescence further showed these CD44⁺ cells were also positive for both of Col XVII and laminin 5 (FIGS. 9b and 9c). Enriched TICs from A549, an anchorage-dependent human lung cancer cell line, increased in suspension survival ability, the PP2A-STAT3-Col XVII-laminin 5 pathway (FIGS. 7k-7m) and the colocalization of Col XVII and laminin 5 (FIG. 9d). An immunodeficient mouse model of malignant pleural effusion was then applied to demonstrate that enriched TICs formed malignant pleural effusion as less as 10³ cells per injection, while cells in monolayer culture only formed malignant pleural effusion with cell numbers greater than 10⁴ cells per injection (FIG. 9e). Moreover, the ability of enriched TICs to form malignant pleural effusion was significantly blocked by specific snRNA against Col XVII or laminin 5 but not by control snRNA (FIG. 9e). These data together suggest the survival of TICs in a genuine suspension condition and the de novo increase in suspension survival by enriched TICs, which was related to the Col XVII-laminin 5 pathway.

### Example 2. Overexpression of Collagen XVII and Laminin-5 Activates Epithelia-to-Mesenchymal Transition (EMT) Via the FAK/AKT/GSK3β Pathway in Lung Cancer Stem-Like Cells (CSCs) and is Associated with Poor Prognosis in Patients with Resectable Lung Cancer

I. Materials and Methods

1. Cell line culture and reagents

The lung cancer cell lines A549 and CL1-1 were obtained from the American Type Culture Collection. Cells were grown in Dulbecco's Modified Eagle Medium (DMEM) (Gibco, Grand Island, N.Y.) containing 10 units/ml penicillin, 10 μg/ml streptomycin, 2 mM glutamine, and 10% fetal bovine serum (FBS; Gibco), in a 37° C. humidified atmosphere with 5% CO₂. For enrichment of cancer stem-like cells (CSCs) in spheroid culture, lung cancer cells were suspended in tumor sphere medium consisting of serum-free DMEM/F12 (Gibco), N2 supplement (Gibco), human recombinant epidermal growth factor (EGF) (20 ng/ml, PeproTech, Rocky Hill, N.J.), and basic fibroblastic growth factor (bFGF) (10 ng/ml, PeproTech). Cell colonies >100 μm in diameter and >50% in area showing 3-dimensional structure and blurred cell margins were defined as spheres. Sphere numbers were counted at day 12 of culture. Cells were harvested and protein lysates were collected for further experiments. Treatment reagents included FAK inhibitor (20 μM) (Calbiochem, San Diego, Calif.; Merk, Schwalbach, Germany), LY294002 (10 μM) (Calbiochem), 1,10-phenanthroline (20 μM) (Sigma-Aldrich; St. Louis, Mo.), PMA (100 nM) (Sigma-Aldrich) and Cyclohexamide (50 μg/ml) (Calbiochem).

2. Antibodies

Antibodies against ADAM9, phospho-AKT (Ser473), β-actin, phospho-FAK (Tyr397), ubiquitin and phospho-GSK3b (Ser9) were purchased from Cell Signaling (Boston, Mass.). Antibodies against laminin-5 (γ72 chain), Sox2, Oct-3/4, Nanog and E-cadherin were from Santa Cruz Biotechnology, Inc. (Santa Cruz, Calif.). Antibodies against fibronectin, vimentin and N-cadherin were purchased from GeneTex (San Antonio, Tex.). Antibodies against Col XVII, Col XVII (NC16A-3) and Snail were purchased from Abcam (Cambridge, Mass.). Antibodies against ADAM10 were from Millipore (Amersham Pharmacia Biotech, Piscataway, N.J.). Antibodies against phospho-Snail (Ser246) were from OriGene Technologies, Inc. (Rockville, Md.).

3. Western Blot Analysis

Cell extracts were prepared with M-PER (Pierce, Rockford, Ill.) plus protease inhibitor cocktail (Halt™; Pierce) and protein concentrations were determined using the bicinchoninic acid (BCA) assay (Pierce). Aliquots of protein lysates were separated on SDS-10% polyacrylamide gels and transferred to PVDF membrane filters, followed by blocking with 5% blotting grade milk (Bio-Rad, Hercules, Calif.) in TBST (20 mM Tris-HCl [pH 7.6], 137 mM NaCl, 1% Tween 20). Membranes were then probed with the indicated primary antibodies, reacted with the corresponding secondary antibodies, and results detected using a chemiluminescence assay (Millipore, Billerica, Mass.). Membranes were exposed to X-ray film to visualize the bands (Amersham Pharmacia Biotech, Piscataway, N.J.).

4. Lentiviral Vector Production and Cell Infection

The shRNA expression plasmids and the bacteria clones for ADAM9 (TRCN46978, TRCN46980), ADAM10 (TRCN6674, TRCN6675), Col XVIIa1 (TRCN118937, TRCN118941) and laminin-5 (TRCN119152, TRCN119156) were provided by the RNAi Core Facility, Academia Sinica (Taipei, Taiwan). Subconfluent tumor cells were infected with lentivirus in the presence of 8 μg/ml polybrene (Sigma-Aldrich). At 24 hours post-infection, media were removed and replaced with fresh growth media containing puromycin (4 μg/ml) to select for infected cells after 48 hours of infection.

5. Migration Assay

Cell migration assay was performed using Boyden chambers. At the end of the assay, cells in the upper chamber and on the upper filter surface were removed, whereas cells on the lower filter surface were fixed with ethanol and stained with Giemsa (Sigma-Aldrich). The number of migrating cells was determined by counting cells in 10 random fields/filter at 200× magnification. Data were calculated as a percentage of migrated cells in the absence of chemoattractant or adherent culture, which was considered as 100%. All experiments were performed in triplicate.

6. Invasion Assay

Cell invasion assays were performed using 24-well BD Biocoat Matrigel Invasion Chambers (Becton, Dickinson and Company, San Jose, Calif.) according to the manufacturer's instructions. In brief, cells were seeded into upper inserts (2.5×10⁴ cells/insert) in DMEM supplemented with 0.1% bovine serum albumin (BSA). Outer wells were filled with DMEM containing 10% FBS as the chemoattractant. After 24 hours of incubation, the membranes with invaded cells were stained with Giemsa (Sigma), washed and mounted on slides. The entire membrane with invading cells was counted by light microscopy. Data are expressed as the number of invaded cells/well. Each assay was performed in triplicate and repeated at least twice.

7. Wound Healing Assay

An in vitro model of the ability of A549 and CL1-1 lung cancer cells to migrate in a monolayer culture was used to measure wound healing ability. Lung cancer cells were seeded into 6-well plates and incubated overnight. The cells were disrupted by scraping them with a 200 IA pipette tip. Migration of cells into wounded areas of the plate was observed at 24 hours. The percent of wounded area filled was calculated as follows: [(mean wound width-mean remaining width)/mean wound width]×100(%).

8. Microarray and Data Analysis

We compared the gene expression pattern after 12 days of A549 lung cancer cells cultured in a spheroid (3D) culture and in a traditional monolayer (2D) culture. Total RNA was isolated with TRIzol reagent (Invitrogen, Carlsbad, Calif.) according to the protocol of the manufacturer. Each sample was processed and analyzed using the Affymetrix Human U133 plus 2.0 array chip (Affymetrix, Santa Clara, Calif.) at the National Microarray and Gene Expression Analysis Core Facility (National Research Program for Genomic Medicine, Taipei, Taiwan). The array data were analyzed using GeneSpring GX v12 software (Agilent Technologies, Santa Clara, Calif.) and classified using Gene Ontology terms.

9. Quantitative Real-Time Polymerase Chain Reaction (PCR)

Total RNA was extracted using TRIzol reagent (Invitrogen, Carlsbad, Calif.) and RNA was reverse-transcribed using Superscript II (Invitrogen) according to the manufacturer's instructions. The samples were analyzed with SYBR Green Master (GeneMark, Georgia Institute of Technology, Atlanta, Ga.) and ABI Step one Real-Time PCR System machine (Applied Biosystems, Carlsbad, Calif.). Specific primers used for PCR are as follows: Col XVIIA1 (forward, 5′-AAAGGACCAATGGGACCACC-3′; reverse, 5′-TTCACCTCTTGGGCCTTGGT-3′).

10. Immunoprecipitation Assay

Aliquots of 500 μg cell lysate were incubated with 2 μg antibody in 500 μl IP Lysis/Wash Buffer (Pierce/Thermo Scientific), with gentle rocking overnight at 4° C., then 25 μl Protein A/G Magnetic beads (Pierce/Thermo Scientific) were added and incubation was continued with gentle rocking for another 2 hours at 4° C. Then, the beads were collected with a magnetic stand and the unbound sample was discarded. The precipitate was washed 2-3 times by adding 500 μl Lysis/Wash Buffer (Pierce/Thermo Scientific), followed by replacement with 500 IA of ultra-pure water. The beads were gently mixed and collected on a magnetic stand, followed by removal of the supernatant and dilution with 50 μl sample buffer. After adequate vortex, the sample was denatured at 100° C. for 10 minutes. The beads were magnetically separated and the supernatant was saved in a new microcentrifuge tube. Immunoblotting was performed with appropriate antibodies.

11. Animal Model of Lung Metastasis

Male Balb/C nude mice were used in the study and maintained as a colony in specific pathogen-free conditions. The mice were used for experiments at 7 weeks of age. Tumor cells A549 (spheroid or non-spheroid culture) without/with Collagen XVII or laminin-5 knockdown (siRNA) (3×10⁵, 5 groups, 4-5 mice in each group) were injected in the tail vein for evaluation of lung metastasis 12 weeks later. The lung tissue of mice was embedded in paraffin and sequentially stained with HE.

12. Patients and Immunohistochemistry

Patients

Ninety-eight patients who underwent surgical resection for lung cancer in Taipei Veterans General Hospital (Taipei, Taiwan) were enrolled in this study. None of these patients received neoadjuvant chemotherapy or radiotherapy. The clinical data including the sex, age, TNM Classification of Malignant Tumors (TNM) status and disease-specific survival were reviewed and calculated. The Institutional Review Board of Taipei Veterans General Hospital approved the protocol for this study (2013-10-030CC).

Immunohistochemistry

Immunohistochemical analysis was performed of Col XVII and laminin-5 in the resected tumor tissues. Paraffin blocks of tumors were cut into 4 μm slices and then processed using standard deparaffinization and rehydration techniques. Anti-Col XVII antibody (1:50) and anti-laminin-5 polycloncal antibody (1:50) were used as the primary antibodies to detect the protein expression Col XVII and laminin-5, respectively. For Col XVII and laminin-5 immunostaining, positive expression was defined as detectable immunoreaction in perinuclear and other cytoplasmic regions of >25% of the cancer cells.

13. Statistical Analysis

Values are shown as the mean (±standard error or deviation) of measurements of at least three independently performed experiments to minimize variation between cell cultures. Student's t-test or one-way ANOVA was employed. Survival curves were calculated using the Kaplan-Meier method and comparisons were performed using the log-rank test. P<0.05 was considered to be statistically significant.

II. Results

1. Lung Cancer Cells in Spheroid Culture have CSC Features and Upregulation of EMT Markers

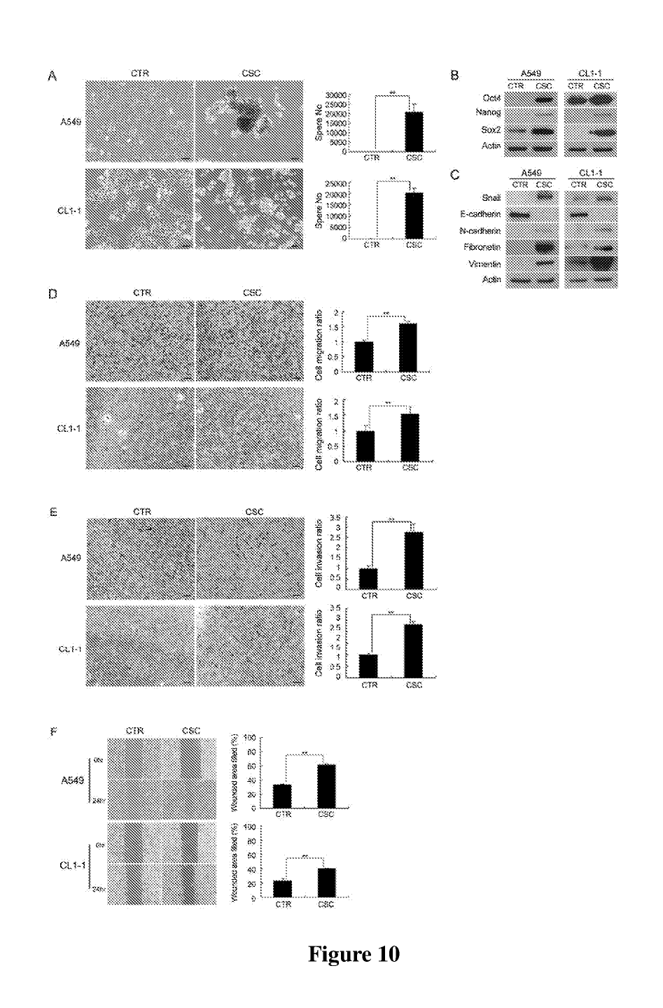

Lung adenocarcinoma A549 and CL1-1 cells cultured in serum-free medium supplemented with EGF and bFGF grew in suspension and started to form spheres in 5 days, and the spheres grew slowly for at least 12 days (FIG. 10A). On the other hand, these lung cancer cells cultured in DMEM supplemented with 10% FBS grew well in monolayer and showed no sphere formation in 12 days. Western blot analysis demonstrated increased expression of the pluripotency markers Nanog, Sox2 and Oct4 in A549 and CL1-1 cells in the spheroid culture (FIG. 10B). In addition, the expression of Snail, the transcription factor involved in the induction of epithelail to mesenchymal transition (EMT), was significantly increased (FIG. 10C). In consistence, the expression of mesenchymal markers N-cadherin, fibronetin and vimentin was upregulated and expression of epithelial marker E-cadherin was downregulated in the spheroid culture (FIG. 10C). A549 and CL1-1 cells grown in spheroid culture demonstrated increased migration (FIG. 10D), invasion (FIG. 10E) and wound closure ability (FIG. 10F) when compared to the cells grown in monolayers. These data suggest that lung cancer cells in spheroid culture exhibited CSC characteristics and displayed EMT phenotypes.

2. Col XVII Upregulated in the Lung Cancer Spheroid Culture is Required for the Maintenance of EMT Phenotypes

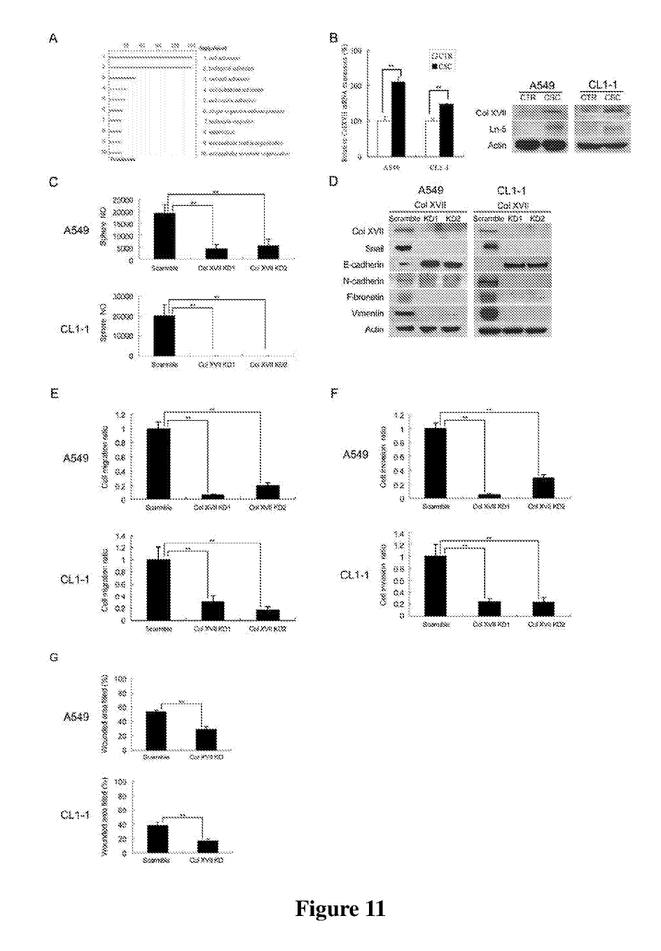

To identify genes responsible for EMT, we performed microarray analysis to establish and compare the expression profiles of lung cancer cells grown in spheroid culture and in monolayer for 12 days. The gene-ontology (GO) analysis revealed that the genes differentially expressed in the spheroid culture were mainly involved in cell adhesion, biological adhesion, cell-cell adhesion, cell-substrate adhesion, cell-matrix adhesion, single-organism cellular process and leukocyte migration (FIG. 11A). Among the genes most upregulated in the cells of spheroid culture (Table 3), we chose Col XVII, a collagenous transmembrane protein and a structural component of the dermoepidermal anchoring complex, for further study based on its involvement in both extracellular adherence and intracellular signaling. To confirm the microarray data, we showed that Col XVII was upregulated at both mRNA and protein levels in A549 and CL1-1 cells when grown in spheroids (FIG. 11B). Loss-of-function approach was taken to demonstrate the involvement of Col XVII in the maintenance of EMT phenotype. As shown in FIG. 11C, Lentivirus-based snRNA knockdown (KD) of Col XVII, using two independent shRNAs targeting Col XVII, both led to significant decrease in sphere formation in A549 and CL1-1 cells. In comparison to the spheroid culture derived from cells harboring the control snRNA, the suspension cells in which Col XVII was knocked down expressed mesenchymal markers Snail, fibronectin, vimentin and N-cadherin at significantly lowered levels, instead they expressed epithelial marker E-cadherin at a much higher level (FIG. 11D). These cells also displayed reduced migratory activity (FIG. 11E), invasiveness (FIG. 11F), and decreased wound closure ability (FIG. 11G). Together, these data suggest that Col XVII was upregulated in lung cancer spheroid cultures and is required for the maintenance of EMT phenotypes.

TABLE 3
The results of microarray analysis in lung cancer cells cultured with or
without spheroid medium

| | | Fold | | | Fold | | | Fold |
|---|---|---|---|---|---|---|---|---|
| | | change | | | change | | | change |
| | | (Daoy- | | | (Daoy- | | | (Daoy- |
| | Entrez | A549 vs. | | Entrez | A549 vs. | Gene | Entrez | A549 vs. |
| Gene Symbol | Gene | A549) | Gene Symbol | Gene | A549) | Symbol | Gene | A549) |
| EPHB3 | 2049 | 3.801267 | SELPLG | 6404 | 4.317229 | RAPH1 | 65059 | 3.9932764 |
| LGALS3BP | 3959 | 3.2198184 | ITGAX | 3687 | 5.878187 | ITGB8 | 3696 | 14.027397 |
| GPNMB | 10457 | 4.010161 | PCDHA1 | 9752 | 3.1180418 | ITGA1 | 3672 | 7.540718 |
| CYP1B1 | 1545 | 3.1746707 | EDA | 1896 | 4.386034 | MPZL3 | 196264 | 4.4793787 |
| CLDN7 | 1366 | 3.4990747 | EDA | 1896 | 4.386034 | LSAMP | 4045 | 4.3488445 |
| ITGB2 | 3689 | 4.004871 | CD84 | 8832 | 5.7293506 | FREM1 | 158326 | 3.1782727 |
| AGT | 183 | 3.4379804 | COL13A1 | 1305 | 6.530901 | CD36 | 948 | 4.027919 |
| CD93 | 22918 | 4.9117465 | RET | 5979 | 9.285199 | LSAMP | 4045 | 4.3488445 |
| S100A8 | 6279 | 7.5469093 | VCAN | 1462 | 3.1193922 | LMLN | 89782 | 8.194991 |
| SOX9 | 6662 | 5.083279 | CADM3 | 57863 | 4.9649873 | NCAM1 | 4684 | 7.178024 |
| SOX9 | 6662 | 5.083279 | TRO | 7216 | 7.238525 | GLDN | 342035 | 9.6214905 |
| NELL2 | 4753 | 12.269872 | FN1 | 2335 | 3.4063003 | F5 | 2153 | 26.5977 |
| TPBG | 7162 | 3.006093 | PCDHGA9 | 56107 | 3.6755307 | TINAG | 27283 | 7.225211 |
| S100A9 | 6280 | 12.334759 | CEACAM1 | 634 | 3.2269318 | PCDHGC4 | 56098 | 7.2429643 |
| DPP4 | 1803 | 11.478296 | CEACAM1 | 634 | 3.2269318 | KIAA1462 | 57608 | 12.404792 |
| ARHGEF17 | 9828 | 4.789358 | COL6A1 | 1291 | 10.630096 | SRCIN1 | 80725 | 3.2516563 |
| COL7A1 | 1294 | 3.3296413 | FN1 | 2335 | 3.4063003 | COL12A1 | 1303 | 8.188657 |
| COL16A1 | 1307 | 3.6154962 | NCAM1 | 4684 | 7.178024 | CADM2 | 253559 | 5.776657 |
| SEBOX | 7448 | 3.326291 | PLXNC1 | 10154 | 6.370429 | RASEF | 158158 | 5.559342 |
| CD22 | 933 | 5.7259383 | MUC5B | 727897 | 19.264805 | FAT3 | 120114 | 4.915558 |
| EPHB3 | 2049 | 3.801267 | SNED1 | 25992 | 5.985906 | NTN1 | 9423 | 9.707547 |
| COL17A1 | 1308 | 3.4681318 | SPON1 | 10418 | 3.2258537 | HEPACAM | 220296 | 16.221796 |
| S1PR1 | 1901 | 3.434874 | CLDN18 | 51208 | 4.3543906 | TPBG | 7162 | 3.006093 |
| F5 | 2153 | 26.5977 | ITGB4 | 3691 | 4.8426313 | COL28A1 | 340267 | 5.9832883 |
| F5 | 2153 | 26.5977 | MUC5AC | 4586 | 154.62657 | HMCN2 | 256158 | 3.957205 |
| DSC2 | 1824 | 7.3692884 | LOC101059911 | 4586 | 8.943722 | RASEF | 158158 | 5.559342 |
| PDPN | 10630 | 5.857203 | FN1 | 2335 | 3.4063003 | RAPH1 | 65059 | 3.9932764 |
| ADAM12 | 8038 | 4.166508 | CDH6 | 1004 | 5.0363927 | SIGLEC11 | 114132 | 11.466388 |
| ITGB4 | 3691 | 4.8426313 | LRRN2 | 10446 | 7.6544204 | PELO | 53918 | 3.3414896 |
| CLDN10 | 9071 | 3.1693237 | FN1 | 2335 | 3.4063003 | RASEF | 158158 | 5.559342 |
| SRPX2 | 27286 | 3.9072075 | COL14A1 | 7373 | 20.285978 | ITGAL | 3683 | 3.438435 |
| CDH6 | 1004 | 5.0363927 | COL4A3 | 1285 | 3.0568228 | CNTNAP4 | 85445 | 4.5087533 |
| DSG3 | 1830 | 6.2298107 | ITGA2B | 3674 | 6.1090703 | CASS4 | 57091 | 3.252114 |
| ANGPT1 | 284 | 3.2186298 | COL7A1 | 1294 | 3.3296413 | CDHR1 | 92211 | 3.9795825 |
| PTPRD | 5789 | 7.0242634 | CD22 | 933 | 5.7259383 | STAB1 | 23166 | 4.913923 |
| HABP2 | 3026 | 5.636392 | NR1D1 | 7067 | 3.0723758 | ITGB2 | 3689 | 4.004871 |
| CD36 | 948 | 4.027919 | CDH8 | 1006 | 9.267938 | FAT3 | 120114 | 4.915558 |
| CDH16 | 1014 | 4.0000186 | CNTNAP2 | 26047 | 6.279269 | ITGAD | 3681 | 5.08718 |
| CEACAM1 | 634 | 3.2269318 | SIRPG | 55423 | 6.263203 | SIGLEC16 | 400709 | 4.9326663 |
| ITGA10 | 8515 | 14.7146845 | PDPK1 | 5170 | 5.9578977 | MAGI1 | 9223 | 3.7478273 |
| EMR2 | 30817 | 3.11195 | CD58 | 965 | 3.3299782 | MAGI1 | 9223 | 3.7478273 |
| CCR8 | 1237 | 7.0967956 | MUC5B | 727897 | 19.264805 | CPXM2 | 119587 | 5.3738723 |
| LOC101060681 | 7146 | 6.6257777 | IL1B | 3553 | 4.036926 | | | |
| PECAM1 | 5175 | 31.220676 | SORBS1 | 10580 | 12.195067 | | | |
| LAMB3 | 3914 | 3.1289375 | ITGA11 | 22801 | 3.3695698 | | | |
| ENTPD1 | 953 | 3.9220545 | PCDHA1 | 9752 | 3.1180418 | | | |
| CEACAM1 | 634 | 3.2269318 | CLDN2 | 9075 | 3.2165232 | | | |
| SEPT5-GP1BB | 100526833 | 7.1952767 | GPR98 | 84059 | 6.1961865 | | | |
| CDH17 | 1015 | 29.525864 | TINAG | 27283 | 7.225211 | | | |
| SPP1 | 6696 | 3.4178011 | RAPH1 | 65059 | 3.9932764 | | | |

3. Laminin-5 May Work Together with Collagen XVII to Support the EMT Phenotypes in the Spheroid Culture of Lung Cancer Cells

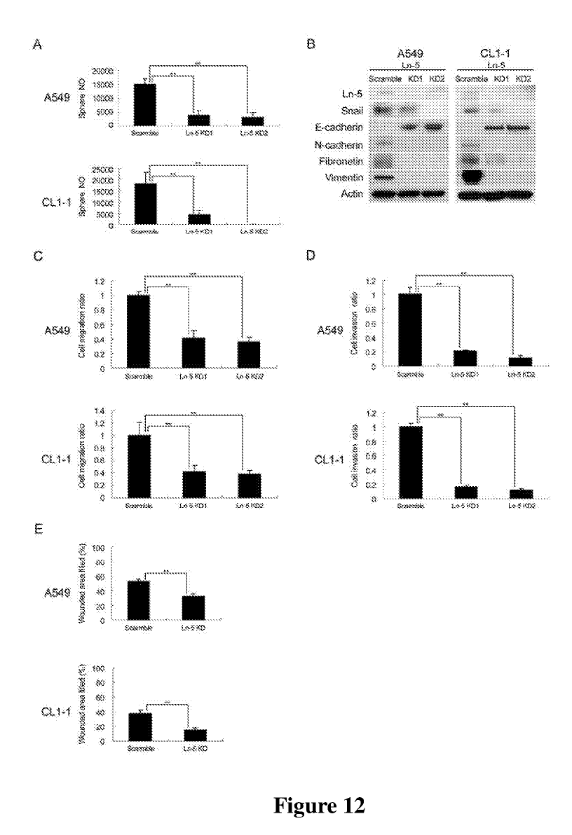

Col XVII has been shown to interact with laminin-5 and, together, participate in cell/matrix interaction. We next examined the involvement of laminin-5 in Col XVII-regulated EMT maintenance in CSCs. We first showed that laminin 5 was upregulated in spheroid culture of A549 and CL1-1 cells as compared to the cells in monolayer (FIG. 11B). A549 and CL1-1 cell clones in which laminin-5 was knocked down and the control KD clones were cultured in spheroid medium. As shown, KD of laminin-5 led to decreased sphere formation (FIG. 12A), which was accompanied with decreased expression of the mesenchymal markers Snail, N-cadherin, fibronectin and vimentin, and increased expression of E-cadherin (FIG. 12B). The Laminin5-KD cells also displayed reduced ability in cell migration (FIG. 12C), invasion (FIG. 12D), and wound closure (FIG. 12E). These data suggest that laminin-5 may work together with Col XVII in support EMT phenotypes in lung cancer in spheroid culture.

4. Shedding of Collagen XVII is Required to Maintain Laminin-5 Expression and EMT Phenotypes in Spheroid Culture of Lung Cancer Cells

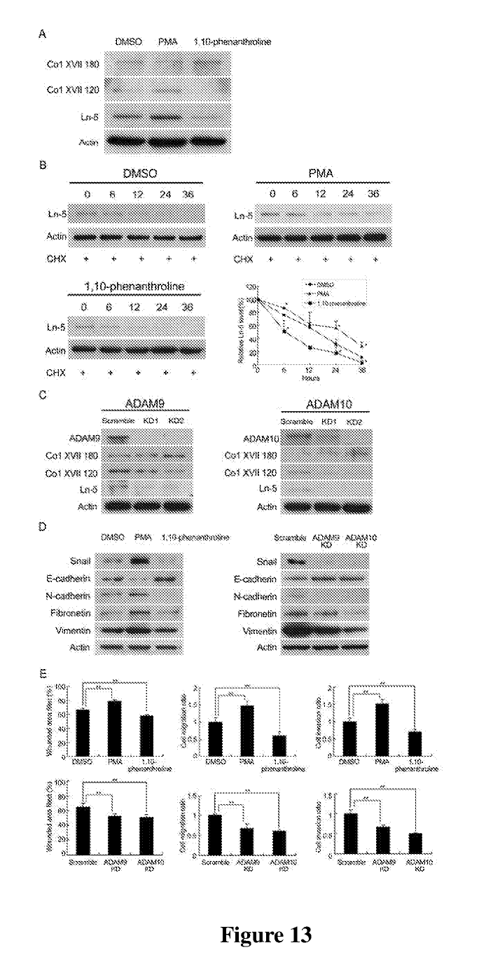

Col XVII has been demonstrated to undergo proteolytic cleavage and the resultant ectodomain of Col XVII was shown to promote keratinocyte motility. We therefore examined whether shedding of Col XVII took place in the spheroid culture. Western blot analysis showed that cells of spheroid culture gave rise to both the full-length Col XVII (180 kDa) and the proteolytic ectodomain fragment (120 kDa) (FIG. 13A, left lane). In contrast, the monolayer cells contained predominantly the full-length protein (data not shown). Incubation of suspension cells in spheroid culture medium in the presence of PMA and 1,10-phenathroline, the promotor and inhibitor of COL XVII shedding, respectively, not only led to increased and decreased shedding of COL XVII, but also positively and negatively regulate the level of laminin 5 (FIG. 13A), suggesting that shedding of Col XVII was involved in the maintenance of laminin-5 protein levels. In support, our data showed that the shedding of Col XVII did not affect the mRNA levels of laminin-5 (data not shown). However, laminin 5 protein exhibited a longer half-life in A549 suspension cells when cultured in spheroid culture medium containing cycloheximide in the presence of PMA, where shedding of Col XVII was promoted (FIG. 13B). On the other hand, laminin 5 was degraded faster in the cells incubated in the presence of 1,10-phenathroline, where shedding of Col XVII was prohibited (FIG. 13B). KD approach was taken to examine the involvement of ADAM9 and ADAM10 in the shedding of Col XVII. Western blot analysis showed decreased levels of Col XVII 120 as well as laminin-5 in A549 cells in which ADAM9 or ADAM10 was individually knocked down (FIG. 13C). In consistence, we also showed that stimulation of Col XVII shedding by PMA increased the expression of mesenchymal markers and decreased that of epithelial marker E-cadherin (FIG. 13D, left panel). In parallel, inhibition of Col XVII shedding by 1,10-phenanthroline or KD of ADAM9 or ADAM10 decreased the expression of mesenchymal markers and increased that of epithelial marker E-cadherin (FIG. 13D, left and right panels). As a consequence, cell migration, invasion and wound closure were enhanced by PMA, and reduced by 1,10-phenanthroline and by KD of ADAM9 or ADAM10 (FIG. 13E). Together, these data suggest that laminin-5 works downstream to mediate Col XVII's effect in support EMT phenotype, and that shedding of Col XVII by ADAMS or ADAM10 is required to stabilize laminin-5, a process required to maintain EMT phenotypes in spheroid culture of lung cancer cells.

5. Spheroid Culture of Lung CSC Metastasize to the Lung in Nude Mice which is Blocked by Depleting the Expression of Col XVII or Laminin-5

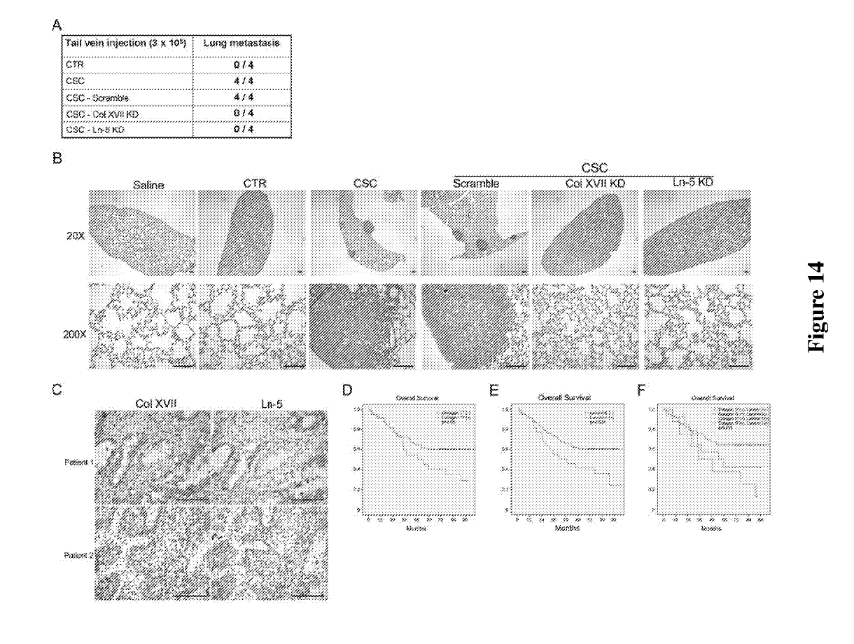

To demonstrate the importance of Col XVII/laminin 5-mediated effect in facilitating EMT phenotypes in spheroid culture of lung cancer cells, we performed the experimental metastasis assay by xenografting the spheroid and monolayer cells into nude mice through tail vein injection. Macro- and microscopic analyses of the lung were performed to assess the formation of metastatic tumors in the lung. s metastasizing to the lung were recorded according to the gross morphology and microscopic analysis of HE stain of the lung sections. As shown in FIG. 14A, inoculation of monolayer cells through tail vein injection did not resulted lung metastasis in 12 weeks. However, inoculation of spheroid cultures of A549 led to tumor formation in the lung in all the animals (FIGS. 14A and 14B). More importantly, KD of Col XVII or laminin 5 completely abolished the ability of the spheroid culture to form lung metastases (FIGS. 14A and 14B). These data suggest the functional role of Col XII and laiminin-5 in promoting tumor metastasis of lung CSCs in vivo.

6. Expression of Col XVII and Laminin-5 Predicts Poorer Prognosis in Patients with Resectable Lung Cancer

We next investigated the clinical significance of Col XVII and laminin-5 expression by immunohistochemical analysis of Col XVII and laminin-5 expression in tumor specimens from a cohort of 98 patients who received lung resection for lung cancer (FIG. 6C). The clinical demographics of 98 patients with lung cancer are shown in Table 2. Kaplan-Meier analysis showed that patients displayed positive staining of either Col XVII (FIG. 14D) or laminin 5 (FIG. 14E) in their tumors exhibited poorer survival as compared to the patients negative for Col XVII and laminin 5 expression. The patients with positive expression of both Col XVII and laminin-5 had the worst prognosis than those with negative expression of Col XVII or laminin-5 (FIG. 14F). These data suggest the usefulness of Col XVII and laminin-5 immunostaining in predicting the prognosis of patients with resectable lung cancer.

7. Col XVII/Laminin 5 Promotes EMT Phenotypes in Spheroid Culture of Lung Cancer Cells Via FAK/AKT/GSK3β Pathway

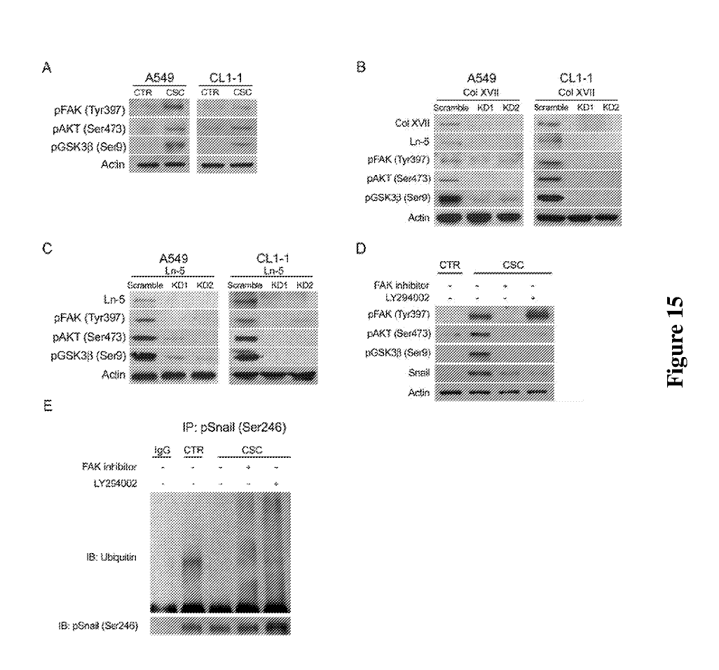

We next explored the mechanism underlying Col XVII/laminin 5-mediated maintenance of EMT phenotypes in CSCs. Snail has been shown as a highly labile protein which undergoes GSK3-regulated phosphorylation and ubiquitin-mediated degradation. We examined whether AKT/GSK3 pathway was involved in Col XVII/laminin 5-mediated Snail upregulation. Western blot analysis showed increased levels of phospho-AKT and phospho-GSK3 in spheroid culture of A549 and CL1-1 cells compared to the monolayer cells (FIG. 15A). In addition, increased level of phospho-FAK was also observed (FIG. 15A). KD of Col XVII resulted in decreased levels of laminin-5, phospho-FAK, phospho-AKT and phospho-GSK3 in A549 and CL1-1 cells in spheroid culture (FIG. 15B). Similarly, KD of laminin-5 also resulted in decreased levels of phospho-FAK, phospho-AKT and phospho-GSK3 in the spheroid culture (FIG. 15C). When A549 cells were cultured in spheroid medium in the presence of LY294002, an inhibitor of PI3-kinase, the phosphorylation of AKT, but not the phosphorylation of FAK, in the spheroid culture was blocked (FIG. 15D), accompanied with decreased levels of phospho-GSK-3 and Snail. Incubation of spheroid cells in the presence of an FAK inhibitor led to reduced levels of phosphor-FMK, phosphor-AKT, and phospho-GSK-3. These data suggest that FAK was downstream of Col XVII/laminin 5 to induce Snail expression via AKT/GSK3 axis. It is shown that ubiquitination of phospho-Snail was readily detected in the monolayer cells, which was completely blocked in the spheroid culture (FIG. 15D). Inhibitors blocking the FAK/AKT/GSK3 signaling axis facilitated Snail phosphorylation and ubiquitin-mediated degradation (FIG. 15E). These data suggest that activation of the FAK/AKT/GSK3 pathway is required for Col XVII/laminin 5-mediated Snail stabilization and maintenance of the EMT phenotype in spheroid culture of lung cancer cells.

8. Col XVII and Laminin 5 Form Organized Structure to Support Suspension Survival.

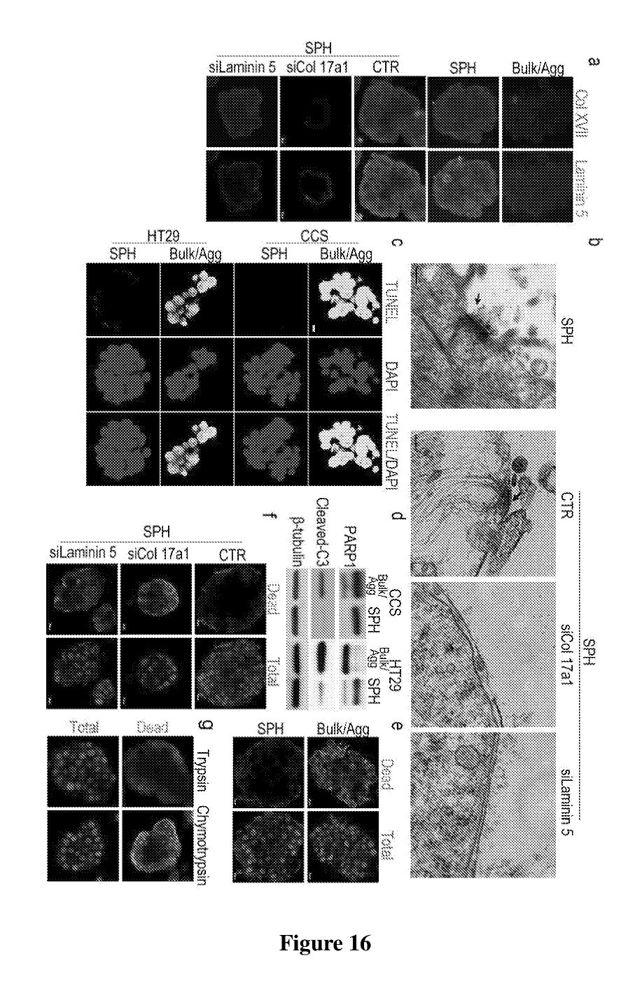

Previous report demonstrates that Col XVII and laminin 5 are components of hemidesmosome for mediating cell adhesion. To exclude the possibility that suspension survival by TICs in spheres was mediated by merely cell aggregation, we demonstrated the expression of Col XVII and laminin 5 (FIG. 16a) and the existence of hemidesmosome-like plaques (FIG. 16b), an organized ECM ultrastructure in the basal lamina of skin epidermis and normal mucosa, in spheres but not in the aggregates of bulk cancer cells formed by hanging drop culture or spheres formed by cancer cells with Col XVII and laminin 5 knockdown (FIG. 16b). When subject to suspension culture for 24 h, the TUNEL staining (FIG. 16c) and the levels of cleaved caspase 3 and PARP1 (FIG. 16d) were greater in aggregates of bulk cancer cells than in enriched TICs. Moreover, live/dead analysis revealed all cells in spheres were live, while only a small number of cells in the center of the aggregates of bulk cancer cells were live (FIG. 16e). Cells with Col XVII or laminin 5 knockdown decreased in the ability to form spheres and most of the cells in spheres were dead when subjected to suspension culture (FIG. 16e), while showing no change in apoptosis when bulk cancer cells were seeded in monolayer culture. Moreover, pre-treatment of the spheres with enzymes that degrade the components of hemidesmosome, such as chymotrypsin²³, but not trypsin, an enzyme that does not degrade hemidesmosome, for 5 min, cause cell death in suspension culture (FIG. 16e). Together, these data suggest Col XVII and laminin 5 form organized structure in spheres, which plays an important role in supporting suspension survival of TICs.

It is believed that a person of ordinary knowledge in the art where the present invention belongs can utilize the present invention to its broadest scope based on the descriptions herein with no need of further illustration. Therefore, the descriptions and claims as provided should be understood as of demonstrative purpose instead of limitative in any way to the scope of the present invention.

1-15. (canceled)

16. A method for preventing tumorigenicity or treating a cancer in a subject which comprises administering to said subject a Collagen XVII (Col XVII) inhibitor in an amount effective to inhibit and prevent survival, tumorigenesis and metastasis of cancer cells and/or cancer stem cells (CSCs).

17. The method of claim 16, wherein the Col XVII is a target of S727-phosphorylated STAT3 (^{S727}STAT3) and responsible for suspension survival and tumor initiation via the activation of the laminin 5-FAK pathway in CSCs.

18. The method of claim 16, wherein the Col XVII inhibitor is a component capable of blocking PP2A-^{S727}STAT3-Col XVII pathway, a component capable of inhibiting Col XVII itself, or a component capable of inhibiting ^{S727}STAT3-Col XVII-laminin 5-FAK pathway whereby mediating suspension survival and tumor initiation in CSCs.

19. The method of claim 18, wherein the Col XVII inhibitor is selected from the group consisting of a microRNA (miRNA), a small interfering RNA (siRNA), a chemical agent, an antibody, an inhibitor of enzyme existing in PP2A-^{S727}STAT3-Col XVII pathway, and an inhibitor of enzyme existing in ^{S727}STAT3-Col XVII-laminin 5-FAK pathway.

20. The method of claim 19, wherein the chemical agent is PP2A activator or STAT3 inhibitor.

21. The method of claim 20, wherein the PP2A activator is forskolin, 1,9-dideoxy-forskolin, or FTY720.

22. The method of claim 20, wherein the STAT3 inhibitor is selected from the group consisting of PY*LKTK, SS 610, S3I-M2001, STA-21, S3I-201, Stattic, IS3 295, CPA-1, CPA-7, Galiellalactone, Peptide aptamers, Decoy ODN, G-quartet ODN, and Peptides.

23. The method of claim 19, wherein the antibody is anti-Col XVII monoclonal or polyclonal antibody, or anti-laminin 5 monoclonal or polyclonal antibody.
